(19) United States
(12) Patent Application Publication
(10) Pub. No.: US 2022/0117915 A1
(43) Pub. Date: Apr. 21, 2022

(54) METHODS OF TREATING ACUTE STRESS DISORDER AND POSTTRAUMATIC STRESS DISORDER

(71) Applicant: Tonix Pharma Holdings Limited, Dublin 2 (IE)

(72) Inventors: Perry Scott Peters, San Diego, CA (US); Gregory M. Sullivan, New York, NY (US); Ernest Mario, Chatham, NJ (US); Herbert Harris, Chapel Hill, NC (US); Seth Lederman, South Dartmouth, MA (US)

(21) Appl. No.: 17/269,106

(22) PCT Filed: Aug. 20, 2019
(86) PCT No.: PCT/IB2019/000940

§ 371 Date: Feb. 17, 2021

Related U.S. Application Data
(60) Provisional application No. 62/720,063, filed on Aug. 20, 2018.

(51) Int. Cl. A61K 31/137; A61P 25/00; A61K 45/06

(52) U.S. Cl. CPC A61K 31/137; A61K 45/06; A61P 25/00

(57) ABSTRACT

This invention relates to methods of treating posttraumatic stress disorder and acute stress disorder using pharmaceutical compositions comprising cyclobenzaprine, amitriptyline, or pharmaceutically acceptable salts thereof. In particular, it relates to methods of treating posttraumatic stress disorder or one or more symptoms thereof in a subject who has experienced a traumatic event less than or equal to about 9 years prior to the commencement or treatment. It also relates to methods of treating acute stress disorder or one or more symptoms thereof in a subject who has experienced a traumatic event less than or equal to about 1 month prior to the commencement of treatment.

This application claims priority to and benefit from U.S. Provisional Patent Application 62/720,063, filed Aug. 20, 2018, the contents and disclosures of which are incorporated herein by reference in their entirety.

### FIELD OF THE DISCLOSURE

This application relates to methods for the treatment of acute stress disorder, posttraumatic stress disorder and associated symptoms thereof. Of particular interest are methods comprising the administration of pharmaceutical compositions comprising cyclobenzaprine, amitriptyline or pharmaceutically acceptable salts thereof to subjects who have experienced a PTSD or ASD causing traumatic event less than or equal to about 9 years prior to the commencement of treatment.

### BACKGROUND OF THE DISCLOSURE

The development of Posttraumatic Stress Disorder (PTSD) is caused by exposure to a traumatic event, and leads to symptoms including difficulty with sleep, nightmares, irritability, difficulty concentrating, hypervigilance, and a persistent exaggerated startle response. Those who suffer from PTSD are at an elevated risk of developing further psychiatric disorders and have a greater risk of suicidal behaviors.

Acute Stress Disorder (ASD) is a disorder in its own right, but it is often a prodromal syndrome that precedes PTSD.

In the pharmacotherapy space, treating ASD or PTSD has been difficult to accomplish. Studies assessing the efficacy of tricyclic antidepressants, monoamine oxidase inhibitors (MAOIs) and serotonin reuptake inhibitors (S SRI) have been conducted in search of a pharmacologic treatment, however, these drugs are generally not effective. For example, in ASD or very early PTSD a study investigating the efficacy of the SSRI escitalopram, commonly used to treat depression, demonstrated that the treatment failed to perform any better than the placebo in preventing development of PTSD (Shalev et al., 2012). Another study evaluated the efficacy of the SSRI paroxetine as a PTSD treatment (Tucker et al., 2001). It was reported that paroxetine offered patients with long-standing PTSD relief (an average of 15 years elapsed since the trauma for these subjects). However, the efficacy of that treatment appeared to be related to the amount of time that had passed since the patient experienced trauma. Specifically, patients who had experienced the trauma more than 5 years prior to the treatment exhibited greater improvement than those whose traumatic experience was more recent. Thus, there is a need to develop an effective pharmacologic treatment that can be offered to patients who have experienced trauma more recently.

Cyclobenzaprine, or 3-(5H-dibenzola[a,d]cyclohepten-5-ylidene)-N,N-dimethyl-1 propanamine, was first approved by the U.S. Food and Drug Administration in 1977 for the treatment of acute muscle spasms of local origin (Katz and Dube, 1988). Subsequent studies have shown that it is a potent serotonergic-2A (5-HT_{2A}) and alpha-adrenergic-1A (α_{1A}) receptorantagonist which improves restorative sleep in neuropsychiatric disorders and fibromyalgia through antagonism of 5-HT_{2A} and α_{1A} receptors during the sleep period (Moldofsky et al., 2011, Moldofsky et al., 2015). The utility of low dose cyclobenzaprine has also been recognized for the treatment of sleep disturbances caused by, exacerbated by, or associated with fibromyalgia syndrome, prolonged fatigue, chronic fatigue, chronic fatigue syndrome, sleep disorders, a psychogenic pain disorders, chronic pain syndrome (type II), the administration of a drug, autoimmune disease, stress or anxiety or for treating an illness caused by or exacerbated by sleep disturbances, and symptoms of such illness and generalized anxiety disorder. See U.S. Pat. Nos. 6,395,788, 6,358,944 and 9,918,948, herein incorporated by reference.

Amitriptyline or 3-(10, 11-dihydro-5H-dibenzo [a,d] cycloheptene-5-ylidene)-N,N-dimethyl-1-propanamine, was first approved by the U.S. Food and Drug Administration for the treatment of depression. Amitriptyline has also been approved for prophylaxis against migraines.

### SUMMARY OF THE DISCLOSURE OF THE APPLICATION

A first aspect of the present disclosure relates to a method for treating post-traumatic stress disorder (PTSD) or one or more symptoms thereof in a subject who has experienced a traumatic event less than or equal to about 9 years prior to the commencement of treatment. In some embodiments, the method comprises administering to the subject a pharmaceutical composition comprising a therapeutically effective amount of cyclobenzaprine, amitriptyline, or pharmaceutically acceptable salts thereof.

A second aspect of the present disclosure relates to a method for treating acute stress disorder (ASD) or one or more symptoms thereof in a subject who has experienced a traumatic event less than or equal to 1 month prior to the commencement of treatment, said method comprising administering to the subject a pharmaceutical composition comprising a therapeutically effective amount of cyclobenzaprine, amitriptyline or pharmaceutically acceptable salts thereof.

Another aspect of the present disclosure relates to a method of treating or preventing PTSD or ASD and associated symptoms in a subject in need thereof, comprising:
a) administering daily to the subject a pharmaceutical composition comprising cyclobenzaprine, amitriptyline or pharmaceutically acceptable salts thereof;
b) assessing the efficacy of the treatment periodically over a course of the treatment;
c) suspending the treatment when the efficacy diminishes;
d) resuming the treatment 4 weeks after suspending the treatment;
   wherein steps (a)-(d) may be repeated one or more times.

A further aspect of the present disclosure relates to method of treating or preventing PTSD and associated symptoms in a subject in need thereof, comprising:
a) administering daily to the subject a pharmaceutical composition comprising cyclobenzaprine, amitriptyline or pharmaceutically acceptable salts thereof;
b) suspending the treatment after about 4 weeks;
c) resuming the treatment about 4 weeks after suspending the treatment;
   wherein steps (a)-(c) may be repeated one or more times.

Still another aspect of this disclosure is a method of determining a therapeutic dosage of cyclobenzaprine or pharmaceutically acceptable salts thereof for the treatment of PTSD or ASD comprising:
a) obtaining a suitable cell or tissue sample from a subject suffering from PTSD or ASD;
b) identifying the CYP1A2, CYP2D6, and CYP3A4 genotype of said subject to determine if the patient has a high cyclobenzaprine metabolizer genotype;
c) assessing the subjects medical history for a history of smoking or use of medications that act as inducers of CYP1A2, CYP2D6 or CYP3A4;
   wherein if the subject has at least one of the criteria identified in step (b) or (c), the dose of cyclobenzaprine administered to the subject is greater than about 5 mg/day;
   wherein if the subject does not have at least one of the criteria identified in step (b) or (c), the dose of cyclobenzaprine administered to the subject is about 5.6 mg/day or less.

Another aspect of this disclosure is a method of determining a therapeutic dosage of amitriptyline or pharmaceutically acceptable salts thereof for the treatment of PTSD or ASD comprising:
a) obtaining a suitable cell or tissue sample from a subject suffering from PTSD or ASD;
b) identifying the CYP1A2, CYP2D6, and CYP3A4 genotype of said subject to determine if the patient has a high amitriptyline metabolizer genotype;
c) assessing the subjects medical history for a history of smoking or use of medications that act as inducers of CYP1A2, CYP2D6 or CYP3A4;
   wherein if the subject has at least one of the criteria identified in step (b) or (c), the dose of amitriptyline administered to the subject is greater than about 11 mg/day;
   wherein if the subject does not have at least one of the criteria identified in step (b) or (c), the dose of amitriptyline administered to the subject is about 11.2 mg/day or less.

Some embodiments of the disclosure are:
1. Use of a pharmaceutical composition comprising a therapeutically effective amount of cyclobenzaprine, amitriptyline, or pharmaceutically acceptable salts thereof for the manufacture of a medicament for the treatment of post-traumatic stress disorder (PTSD) or one or more symptoms thereof in a subject who has experienced a traumatic event less than or equal to about 9 years prior to the commencement of said treatment.
2. Use of a pharmaceutical composition comprising a therapeutically effective amount of cyclobenzaprine, amitriptyline or pharmaceutically acceptable salts thereof for the manufacture of a medicament for the treatment of acute stress disorder (ASD) or one or more symptoms thereof in a subject who has experienced a traumatic event less than or equal to 1 month prior to the commencement of said treatment.
3. The use of embodiment 1 or 2, wherein the traumatic event is a criterion A traumatic event.
4. The use of any one of embodiments 1-3, wherein the medicament is for administration is once daily.
5. The use of any one of embodiments 1-4, wherein the treatment does not exceed 4 weeks.
6. The use of any one of embodiments 2, or 3-5 as they depend from embodiment 2, wherein the treatment of ASD alleviates the development of PTSD and associated symptoms thereof in the subject.
7. The use of any one of embodiments 1-6, wherein cyclobenzaprine or amitriptyline is a free base.
8. The use of any one of embodiments 1-6, wherein cyclobenzaprine or amitriptyline is a pharmaceutically acceptable salt thereof.
9. The use of any one of embodiments 1-8, wherein the medicament is formulated for sublingual, buccal, oral, suppository, intravenous, intramuscular, subcutaneous, inhalational, intranasal, transdermal, parenteral, rectal, or vaginal administration.
10. The use of embodiment 9, wherein the medicament is formulated for sublingual administration.
11. The use of any one of embodiments 1-10, wherein the medicament comprises a basifying agent.
12. The use of embodiment 11, wherein the basifying agent is selected from the group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate.
13. The use of any one of embodiments 1-12, wherein the efficacy of the treatment increases with decreasing time between the commencement of treatment and the traumatic event.
14. The use of any one of embodiments 1-13, wherein the amount of cyclobenzaprine, or pharmaceutically acceptable salts thereof that is administered is between about 0.1 mg and about 50 mg/day.
15. The use of embodiment 14, wherein the amount of cyclobenzaprine or pharmaceutically acceptable salt thereof that is administered is between about 0.5 mg and about 30 mg/day.
16. The use of embodiment 15, where the amount of cyclobenzaprine or pharmaceutically acceptable salt thereof administered is between about 1 mg and about 20 mg/day.
17. The use of any one of embodiments 1-13, wherein the amount of amitriptyline, or pharmaceutically acceptable salt thereof that is administered is between about 0.1 mg and about 150 mg/day.
18. The use of embodiment 17, wherein the amount of amitriptyline or pharmaceutically acceptable salt thereof that is administered is between about 1.0 mg and about 90 mg/day.
19. The use of embodiment 18, where the amount of amitriptyline or pharmaceutically acceptable salt thereof that is administered is between about 3 mg and about 60 mg/day.
20. The use of embodiment 1 or 2, wherein the medicament is for sequential or concurrent administration with a compound selected from the group consisting of an alpha-1-adrenergic receptor antagonist, a beta-adrenergic antagonist, an anticonvulsant, a selective serotonin reuptake inhibitor and a serotonin-norepinephrine reuptake inhibitor.
21. The use of embodiment 20, wherein the alpha-1-adrenergic receptor antagonist is prazosin.
22. The use of embodiment 20, wherein the selective serotonin reuptake inhibitor is sertraline, paroxetine, fluoxetine, citalopram or escitalopram.
23. The use of embodiment 1 or 2, wherein the medicament is for administration in combination with psychotherapeutic intervention during the course of treatment.
24. The use of any one of embodiments 1 or 3-5 or 7-23 as they depend from embodiment 1, wherein at least one of the symptoms of PTSD is eliminated or ameliorated.
25. The use of embodiment 24 wherein the symptoms of PTSD are selected from the group consisting of intrusion symptoms, avoidance symptoms, cognition and mood symptoms, arousal and reactivity symptoms, difficulty falling sleep, irritability, difficulty concentrating, hypervigilance, and persistent exaggerated startle response.
26. The use of any one of embodiments 2, or 3-23 as they depend from embodiment 2, wherein at least one of the symptoms of ASD is eliminated or ameliorated.
27. The use of embodiment 26 wherein the symptoms of ASD are selected from the group consisting of reexperiencing symptoms, avoidance symptoms, arousal symptoms, difficulty with sleep, nightmares, irritability, difficulty concentrating, hypervigilance, persistent exaggerated startle response, feelings such as not knowing where you are, and feeling as if you are outside of your body.
28. The use of embodiment 1, wherein the medicament is for administration during the rapid recovery phase, the remitting phase, or the persistent phase of PTSD.
29. Use of a pharmaceutical composition comprising cyclobenzaprine, amitriptyline or pharmaceutically acceptable salts thereof for the manufacture of a medicament for the treatment or prevention of PTSD, ASD or one or more associated symptoms thereof in a subject in need thereof, wherein the treatment comprises:
   a) administering the medicament daily to the subject;
   b) assessing the efficacy of the treatment periodically over a course of the treatment;
   c) suspending the administration of the medicament when the efficacy diminishes;
   d) resuming the administration of the medicament 4 weeks after suspending the treatment;
   wherein steps (a)-(d) may be repeated one or more times.
30. Use of a pharmaceutical composition comprising cyclobenzaprine, amitriptyline or pharmaceutically acceptable salts thereof for the manufacture of a medicament for the treatment or prevention of PTSD, ASD or one or more associated symptoms thereof in a subject in need thereof, wherein the treatment comprises:
   a) administering the medicament daily to the subject;
   b) suspending the administration after about 4 weeks;
   c) resuming the administration about 4 weeks after suspending the administration;
   wherein steps (a)-(c) may be repeated one or more times.
31. The use of embodiment 29 or 30, wherein the treatment or prevention is of PTSD, and the subject has experienced a traumatic event less than or equal to about 9 years prior to the commencement of treatment.
32. The use of embodiment 31, wherein the efficacy of the treatment is measured at least about every 2 weeks after the treatment begins.
33. The use of embodiment 32, wherein the efficacy of the treatment is assessed based on the subject's Clinician Administered PTSD Scale for DSM-5 (CAPS-5) score.
34. The use of any one of embodiments 29-33, wherein the cyclobenzaprine or amitriptyline in the pharmaceutical composition is the cyclobenzaprine or amitrptyline free base.
35. The use of any one of embodiments 29-33, wherein the cyclobenzaprine or amitriptyline in the pharmaceutical composition is a pharmaceutically acceptable cyclobenzaprine or amitriptyline salt.
36. The use of any one of embodiments 29-35, wherein the medicament is administered sublingually, buccally, orally, in a suppository, intravenously, intramuscularly, subcutaneously, inhalationally, intranasally, in a thin film, transdermally, parenterally, rectally, or vaginally.
37. The use of embodiment 36, wherein the medicament is administered sublingually.
38. The use of any one of embodiments 29-37, wherein the pharmaceutical composition comprises a basifying agent.
39. The use of embodiment 38, wherein the basifying agent is selected from the group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate.
40. The use of any one of embodiments 29-39, wherein the efficacy of the treatment increases with decreasing time between the commencement of treatment and the traumatic event.
41. The use of any one of embodiments 29-40, wherein the amount of cyclobenzaprine, amitriptyline or pharmaceutically acceptable salts thereof that is in the medicament is between about 0.1 mg and about 50 mg/day.
42. The use of embodiment 41, wherein the amount of cyclobenzaprine or pharmaceutically acceptable salt thereof that is in the medicament is between about 0.5 mg and about 30 mg/day.
43. The use of embodiment 42, where the amount of cyclobenzaprine, amitriptyline or pharmaceutically acceptable salts thereof that is in the medicament is between about 1 mg and about 20 mg/day.
44. The use of embodiment 29-40, wherein the amount of amitriptyline or pharmaceutically acceptable salt thereof that is in the medicament is between about 0.1 mg and about 150 mg/day.
45. The use of embodiment 44, wherein the amount of amitriptyline or pharmaceutically acceptable salt thereof that is in the medicament is between about 1.0 mg and about 90 mg/day.
46. The use of embodiment 45, where the amount of amitriptyline or pharmaceutically acceptable salt thereof that is in the medicament is between about 3 mg and about 60 mg/day.
47. The use of embodiment 29 or 30, wherein the medicament is for sequential or concurrent administration in combination with a compound selected from the group consisting of an alpha-1-adrenergic receptor antagonist, a beta-adrenergic antagonist, an anticonvulsant, a selective serotonin reuptake inhibitor and a serotonin-norepinephrine reuptake inhibitor.
48. The use of embodiment 47, wherein the alpha-1-adrenergic receptor antagonist is prazosin.
49. The use of embodiment 47, wherein the selective serotonin reuptake inhibitor is sertraline, paroxetine, fluoxetine, citalopram or escitalopram.
50. The use of embodiment 29 or 30, wherein the medicament is administered in combination with psychotherapeutic intervention during the course of treatment.
51. The use of any one of embodiments 29-50, wherein the treatment or prevention is of PTSD, and at least one of the symptoms of PTSD are eliminated or ameliorated.
52. The use of embodiment 51 wherein the symptoms of PTSD are selected from the group consisting of intrusion symptoms, avoidance symptoms, cognition and mood symptoms, arousal and reactivity symptoms, difficulty falling sleep, irritability, difficulty concentrating, hypervigilance, and persistent exaggerated startle response.
53. The use of embodiment 29 or 30, wherein the subject has experienced a criterion A trauma.
54. The use of embodiment 53, wherein the criterion A trauma results in ASD or symptoms thereof
55. The use of embodiment 54, wherein at least one of the symptoms of ASD are eliminated or ameliorated.
56. The use of embodiment 55, wherein the symptoms of ASD are selected from the group consisting of reexperiencing symptoms, avoidance symptoms, arousal symptoms, difficulty with sleep, nightmares, irritability, difficulty concentrating, hypervigilance, persistent exaggerated startle response, feelings such as not knowing where you are, and feeling as if you are outside of your body.
57. A method for treating post-traumatic stress disorder (PTSD) or one or more symptoms thereof in a subject who has experienced a traumatic event less than or equal to about 9 years prior to the commencement of treatment, said method comprising administering to the subject, a pharmaceutical composition comprising a therapeutically effective amount of cyclobenzaprine, amitriptyline, or pharmaceutically acceptable salts thereof.
58. A method for treating acute stress disorder (ASD) or one or more symptoms thereof in a subject who has experienced a traumatic event less than or equal to 1 month prior to the commencement of treatment, said method comprising administering to the subject a pharmaceutical composition comprising a therapeutically effective amount of cyclobenzaprine, amitriptyline or pharmaceutically acceptable salts thereof.
59. The method of embodiment 57 or 58, wherein the traumatic event is a criterion A traumatic event.
60. The method of any one of embodiments 57-59, wherein the pharmaceutical composition is administered once daily.
61. The method of any one of embodiments 57-60, wherein the treatment does not exceed 4 weeks.
62. The method of any one of embodiments 58, or 59-61 as they depend from embodiment 58, wherein the treatment of ASD alleviates the development of PTSD and associated symptoms thereof in the subject.
63. The method of any one of embodiments 57-62, wherein cyclobenzaprine or amitriptyline is administered as a free base.
64. The method of any one of 57-62, wherein cyclobenzaprine or amitriptyline is administered as a pharmaceutically acceptable salt thereof.
65. The method of any one of embodiments 57-64, wherein the pharmaceutical composition is administered sublingually, buccally, orally, in a suppository, intravenously, intramuscularly, subcutaneously, inhalationally, intranasally, in a thin film, transdermally, parenterally, rectally, or vaginally.
66. The method of embodiment 65, wherein the pharmaceutical composition is administered sublingually.
67. The method of any one of embodiments 57-66, wherein the pharmaceutical composition comprises a basifying agent.
68. The method of embodiment 67, wherein the basifying agent is selected from the group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate.
69. The method of any one of embodiments 57-68, wherein the efficacy of the treatment increases with decreasing time between the commencement of treatment and the traumatic event.
70. The method of any one of embodiments 57-69, wherein the amount of cyclobenzaprine, or pharmaceutically acceptable salts thereof that is administered is between about 0.1 mg and about 50 mg/day.
71. The method of embodiment 70, wherein the amount of cyclobenzaprine or pharmaceutically acceptable salt thereof that is administered is between about 0.5 mg and about 30 mg/day.
72. The method of embodiment 71, where the amount of cyclobenzaprine or pharmaceutically acceptable salt thereof administered is between about 1 mg and about 20 mg/day.
73. The method of any one of embodiments 57-69, wherein the amount of amitriptyline, or pharmaceutically acceptable salt thereof that is administered is between about 0.1 mg and about 150 mg/day.
74. The method of embodiment 73, wherein the amount of amitriptyline or pharmaceutically acceptable salt thereof that is administered is between about 1.0 mg and about 90 mg/day.
75. The method of embodiment 74, where the amount of amitriptyline or pharmaceutically acceptable salt thereof that is administered is between about 3 mg and about 60 mg/day.
76. The method of embodiment 57 or 58, wherein the method further comprises administering sequentially to or concurrently with the cyclobenzaprine, amitriptyline or pharmaceutically acceptable salts thereof, a compound selected from the group consisting of an alpha-1-adrenergic receptor antagonist, a beta-adrenergic antagonist, an anticonvulsant, a selective serotonin reuptake inhibitor and a serotonin-norepinephrine reuptake inhibitor.
77. The method of embodiment 76 wherein the alpha-1-adrenergic receptor antagonist is prazosin.
78. The method of embodiment 76, wherein the selective serotonin reuptake inhibitor is sertraline, paroxetine, fluoxetine, citalopram or escitalopram.
79. The method of embodiment 57 or 58, also comprising psychotherapeutic intervention during the course of treatment.
80. The method of any one of embodiments 57 or 59-61 or 63-79 as they depend from embodiment 57, wherein at least one of the symptoms of PTSD is eliminated or ameliorated.
81. The method of embodiment 80 wherein the symptoms of PTSD are selected from the group consisting of intrusion symptoms, avoidance symptoms, cognition and mood symptoms, arousal and reactivity symptoms, difficulty falling sleep, irritability, difficulty concentrating, hypervigilance, and persistent exaggerated startle response.
82. The method of any one of embodiments 58, or 59-79 as they depend from embodiment 58, wherein at least one of the symptoms of ASD is eliminated or ameliorated.
83. The method of embodiment 82 wherein the symptoms of ASD are selected from the group consisting of reexperiencing symptoms, avoidance symptoms, arousal symptoms, difficulty with sleep, nightmares, irritability, difficulty concentrating, hypervigilance, persistent exaggerated startle response, feelings such as not knowing where you are, and feeling as if you are outside of your body.
84. The method of embodiment 57, wherein the treatment is administered during the rapid recovery phase, the remitting phase, or the persistent phase of PTSD.
85. A method of treating or preventing PTSD, ASD or one or more associated symptoms thereof in a subject in need thereof, comprising:
   a) administering daily to the subject a pharmaceutical composition comprising cyclobenzaprine, amitriptyline or pharmaceutically acceptable salts thereof;
   b) assessing the efficacy of the treatment periodically over a course of the treatment;
   c) suspending the treatment when the efficacy diminishes;
   d) resuming the treatment 4 weeks after suspending the treatment;
   wherein steps (a)-(d) may be repeated one or more times.
86. A method of treating or preventing PTSD, ASD or one or more associated symptoms thereof in a subject in need thereof, comprising:
   a) administering daily to the subject a pharmaceutical composition comprising cyclobenzaprine, amitriptyline or pharmaceutically acceptable salts thereof;
   b) suspending the treatment after about 4 weeks;
   c) resuming the treatment about 4 weeks after suspending the treatment;
   wherein steps (a)-(c) may be repeated one or more times.
87. The method of embodiment 85 or 86, wherein the treatment or prevention is of PTSD, and the subject has experienced a traumatic event less than or equal to about 9 years prior to the commencement of treatment.
88. The method of embodiment 87, wherein the efficacy of the treatment is measured at least about every 2 weeks after the treatment begins.
89. The method of embodiment 88, wherein the efficacy of the treatment is assessed based on the subject's Clinician Administered PTSD Scale for DSM-5 (CAPS-5) score.
90. The method of any one of embodiments 85-89, wherein cyclobenzaprine or amitriptyline is administered as a free base.
91. The method of any one of embodiments 85-89, wherein cyclobenzaprine or amitriptyline is administered as a pharmaceutically acceptable salt thereof.
92. The method of any one of embodiments 85-91, wherein the pharmaceutical composition is administered sublingually, buccally, orally, in a suppository, intravenously, intramuscularly, subcutaneously, inhalationally, intranasally, in a thin film, transdermally, parenterally, rectally, or vaginally.
93. The method of embodiment 92, wherein the pharmaceutical composition is administered sublingually.
94. The method of any one of embodiments 85-93, wherein the pharmaceutical composition comprises a basifying agent.
95. The method of embodiment 94, wherein the basifying agent is selected from the group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate.
96. The method of any one of embodiments 85-95, wherein the efficacy of the treatment increases with decreasing time between the commencement of treatment and the traumatic event.
97. The method of any one of embodiments 85-96, wherein the amount of cyclobenzaprine, amitriptyline or pharmaceutically acceptable salts thereof that is administered is between about 0.1 mg and about 50 mg/day.
98. The method of embodiment 97, wherein the amount of cyclobenzaprine or pharmaceutically acceptable salt thereof that is administered is between about 0.5 mg and about 30 mg/day.
99. The method of embodiment 98, where the amount of cyclobenzaprine, amitriptyline or pharmaceutically acceptable salts thereof that is administered is between about 1 mg and about 20 mg/day.
100. The method of any one of embodiments 85-96, wherein the amount of amitriptyline or pharmaceutically acceptable salt thereof that is administered is between about 0.1 mg and about 150 mg/day.
101. The method of embodiment 100, wherein the amount of amitriptyline or pharmaceutically acceptable salt thereof that is administered is between about 1.0 mg and about 90 mg/day.
102. The method of embodiment 101, where the amount of amitriptyline or pharmaceutically acceptable salt thereof that is administered is between about 3 mg and about 60 mg/day.
103. The method of any one of embodiments 85-102, wherein the method further comprises administering sequentially to or concurrently with cyclobenzaprine, amitriptyline or pharmaceutically acceptable salts thereof, a compound selected from the group consisting of an alpha-1-adrenergic receptor antagonist, a beta-adrenergic antagonist, an anticonvulsant, a selective serotonin reuptake inhibitor and a serotonin-norepinephrine reuptake inhibitor.
104. The method of embodiment 103, wherein the alpha-1-adrenergic receptor antagonist is prazosin.
105. The method of embodiment 103, wherein the selective serotonin reuptake inhibitor is sertraline, paroxetine, fluoxetine, citalopram or escitalopram.
106. The method of embodiment 85 or 86, wherein the pharmaceutical composition is administered in combination with psychotherapeutic intervention during the course of treatment.
107. The method of any one of embodiments 85-106, wherein the treatment or prevention is of PTSD, and at least one of the symptoms of PTSD are eliminated or ameliorated.
108. The method of embodiment 107 wherein the symptoms of PTSD are selected from the group consisting of intrusion symptoms, avoidance symptoms, cognition and mood symptoms, arousal and reactivity symptoms, difficulty falling sleep, irritability, difficulty concentrating, hypervigilance, and persistent exaggerated startle response.
109. The method of embodiment 85 or 86, wherein the subject has experienced a criterion A trauma.
110. The method of embodiment 109, wherein the criterion A trauma results in ASD or symptoms thereof
111. The method of embodiment 110, wherein at least one of the symptoms of ASD are eliminated or ameliorated.
112. The method of embodiment 111, wherein the symptoms of ASD are selected from the group consisting of reexperiencing symptoms, avoidance symptoms, arousal symptoms, difficulty with sleep, nightmares, irritability, difficulty concentrating, hypervigilance, persistent exaggerated startle response, feelings such as not knowing where you are, and feeling as if you are outside of your body.
113. A method of determining a therapeutic dosage of cyclobenzaprine or pharmaceutically acceptable salts thereof, for the treatment of PTSD or ASD comprising:
   a) obtaining a suitable cell or tissue sample from a subject suffering from PTSD or ASD;
   b) identifying the CYP1A2, CYP2D6, and CYP3A4 genotype of said subject to determine if the patient has a high cyclobenzaprine metabolizer genotype;
   c) assessing the subject's medical history for a history of smoking or use of medications that act as inducers of CYP3A4;
   wherein if the subject has at least one of the criteria identified in step (b) or (c), the dose of cyclobenzaprine administered to the subject is greater than about 5 mg/day;
   wherein if the subject does not have at least one of the criteria identified in step (b) or (c), the dose of cyclobenzaprine administered to the subject is about 5.6 mg/day or less.
114. A method of determining a therapeutic dosage of amitriptyline or pharmaceutically acceptable salts thereof for the treatment of PTSD or ASD comprising:
   a) obtaining a suitable cell or tissue sample from a subject suffering from PTSD or ASD;
   b) identifying the CYP1A2, CYP2D6, and CYP3A4 genotype of said subject to determine if the patient has a high amitriptyline metabolizer genotype;
   c) assessing the subject's medical history for a history of smoking or use of medications that act as inducers of CYP3A4;
   wherein if the subject has at least one of the criteria identified in step (b) or (c), the dose of amitriptyline administered to the subject is greater than about 11 mg/day;
   wherein if the subject does not have at least one of the criteria identified in step (b) or (c), the dose of amitriptyline administered to the subject is about 11.2 mg/day or less.
115. The method of embodiment 113 or 114, wherein the medications that act as inducers of CYP3A4 are selected from carbamazepine, phenytoin, phenobarbital, and nevirapine.
116. The method of embodiment 113 or 114, wherein the treatment is of PTSD, and the subject has experienced a traumatic event less than or equal to about 9 years prior to the commencement of treatment.
117. The method of embodiment 113 or 114, wherein the cyclobenzaprine, amitriptyline or pharmaceutically acceptable salts thereof is administered as a pharmaceutical composition.
118. The method of embodiment 117, wherein the pharmaceutical composition comprises cyclobenzaprine or amitriptyline free base.
119. The method of embodiment 117, wherein the pharmaceutical composition comprises a pharmaceutically acceptable salt of cyclobenzaprine or amitriptyline.
120. The method of any one of embodiments 117-119, wherein the pharmaceutical composition is administered sublingually, buccally, orally, in a suppository, intravenously, intramuscularly, subcutaneously, inhalationally, intranasally, in a thin film, transdermally, parenterally, rectally, or vaginally.
121. The method of embodiment 120, wherein the pharmaceutical composition is administered sublingually.
122. The method of any one of embodiments 117-121, wherein the pharmaceutical composition comprises a basifying agent.
123. The method of embodiment 122, wherein the basifying agent is selected from the group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate.
124. The method of any one of embodiments 113-123, wherein the cyclobenzaprine, amitriptyline or pharmaceutically acceptable salts thereof is administered sequentially to or concurrently with a compound selected from the group consisting of an alpha-1-adrenergic receptor antagonist, a beta-adrenergic antagonist, an anticonvulsant, a selective serotonin reuptake inhibitor and a serotonin-norepinephrine reuptake inhibitor.
125. The method of embodiment 124, wherein the alpha-1-adrenergic receptor antagonist is prazosin.
126. The method of embodiment 124, wherein the selective serotonin reuptake inhibitor is sertraline, paroxetine, fluoxetine, citalopram or escitalopram.
127. The method of any one of embodiment 117-126, wherein the pharmaceutical composition is administered in combination with psychotherapeutic intervention during the course of treatment.
128. The method of any one of embodiment 113-127, wherein at least one of the symptoms of PTSD are eliminated or ameliorated.
129. The method of embodiment 128, wherein the symptoms of PTSD are selected from the group consisting of intrusion symptoms, avoidance symptoms, cognition and mood symptoms, arousal and reactivity symptoms, difficulty falling sleep, irritability, difficulty concentrating, hypervigilance, and persistent exaggerated startle response.
130. The method of any one of embodiments 113-127, wherein at least one of the symptoms of ASD are eliminated or ameliorated.
131. The method of embodiment 130 wherein the symptoms of ASD are selected from the group consisting of reexperiencing symptoms, avoidance symptoms, arousal symptoms, difficulty with sleep, nightmares, irritability, difficulty concentrating, hypervigilance, persistent exaggerated startle response, feelings such as not knowing where you are, and feeling as if you are outside of your body.
132. A pharmaceutical composition comprising a therapeutically effective amount of cyclobenzaprine, amitriptyline, or pharmaceutically acceptable salts thereof for the treatment of post-traumatic stress disorder (PTSD) or one or more symptoms thereof in a subject who has experienced a traumatic event less than or equal to about 9 years prior to the commencement of said treatment.
133. A pharmaceutical composition comprising a therapeutically effective amount of cyclobenzaprine, amitriptyline or pharmaceutically acceptable salts thereof for the treatment of acute stress disorder (ASD) or one or more symptoms thereof in a subject who has experienced a traumatic event less than or equal to 1 month prior to the commencement of said treatment.
134. The pharmaceutical composition of embodiment 132 or 133, wherein the traumatic event is a criterion A traumatic event.
135. The pharmaceutical composition of any one of embodiments 132-134, wherein the medicament is for administration is once daily.
136. The pharmaceutical composition of any one of embodiments 132-135, wherein the treatment does not exceed 4 weeks.
137. The pharmaceutical composition of any one of embodiments 133, or 134-136 as they depend from embodiment 133, wherein the treatment of ASD alleviates the development of PTSD and associated symptoms thereof in the subject.
138. The pharmaceutical composition of any one of embodiments 132-137, wherein cyclobenzaprine or amitriptyline is a free base.
139. The pharmaceutical composition of any one of embodiments 132-137, wherein cyclobenzaprine or amitriptyline is a pharmaceutically acceptable salt thereof.
140. The pharmaceutical composition of any one of embodiments 132-139, wherein the pharmaceutical composition is formulated for sublingual, buccal, oral, suppository, intravenous, intramuscular, subcutaneous, inhalational, intranasal, transdermal, parenteral, rectal, or vaginal administration.
141. The pharmaceutical composition of embodiment 140, wherein the pharmaceutical composition is formulated for sublingual administration.
142. The pharmaceutical composition of any one of embodiments 132-141, wherein the pharmaceutical composition comprises a basifying agent.
143. The pharmaceutical composition of embodiment 142, wherein the basifying agent is selected from the group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate.
144. The pharmaceutical composition of any one of embodiments 132-143, wherein the efficacy of the treatment increases with decreasing time between the commencement of treatment and the traumatic event.
145. The pharmaceutical composition of any one of embodiments 132-144, wherein the amount of cyclobenzaprine, or pharmaceutically acceptable salts thereof that is administered is between about 0.1 mg and about 50 mg/day.
146. The pharmaceutical composition of embodiment 145, wherein the amount of cyclobenzaprine or pharmaceutically acceptable salt thereof that is administered is between about 0.5 mg and about 30 mg/day.
147. The pharmaceutical composition of embodiment 146, where the amount of cyclobenzaprine or pharmaceutically acceptable salt thereof administered is between about 1 mg and about 20 mg/day.
148. The pharmaceutical composition of any one of embodiments 132-144, wherein the amount of amitriptyline, or pharmaceutically acceptable salt thereof that is administered is between about 0.1 mg and about 150 mg/day.
149. The pharmaceutical composition of embodiment 148, wherein the amount of amitriptyline or pharmaceutically acceptable salt thereof that is administered is between about 1.0 mg and about 90 mg/day.
150. The pharmaceutical composition of embodiment 149, where the amount of amitriptyline or pharmaceutically acceptable salt thereof that is administered is between about 3 mg and about 60 mg/day.
151. The pharmaceutical composition of embodiment 132 or 133, wherein a compound selected from the group consisting of an alpha-1-adrenergic receptor antagonist, a beta-adrenergic antagonist, an anticonvulsant, a selective serotonin reuptake inhibitor and a serotonin-norepinephrine reuptake inhibitor is administered sequentially to or concurrently with the pharmaceutical composition.
152. The pharmaceutical composition of embodiment 151, wherein the alpha-1-adrenergic receptor antagonist is prazosin.
153. The pharmaceutical composition of embodiment 151, wherein the selective serotonin reuptake inhibitor is sertraline, paroxetine, fluoxetine, citalopram or escitalopram.
154. The pharmaceutical composition of embodiment 132 or 133, wherein the pharmaceutical composition is for administration in combination with psychotherapeutic intervention during the course of treatment.
155. The pharmaceutical composition of any one of embodiments 132 or 134-136 or 138-154 as they depend from embodiment 132, wherein at least one of the symptoms of PTSD is eliminated or ameliorated.
156. The use of embodiment 155 wherein the symptoms of PTSD are selected from the group consisting of intrusion symptoms, avoidance symptoms, cognition and mood symptoms, arousal and reactivity symptoms, difficulty falling sleep, irritability, difficulty concentrating, hypervigilance, and persistent exaggerated startle response.
157. The use of any one of embodiments 133, or 134-154 as they depend from embodiment 134, wherein at least one of the symptoms of ASD is eliminated or ameliorated.
158. The use of embodiment 157 wherein the symptoms of ASD are selected from the group consisting of reexperiencing symptoms, avoidance symptoms, arousal symptoms, difficulty with sleep, nightmares, irritability, difficulty concentrating, hypervigilance, persistent exaggerated startle response, feelings such as not knowing where you are, and feeling as if you are outside of your body.
159. The use of embodiment 132, wherein the medicament is for administration during the rapid recovery phase, the remitting phase, or the persistent phase of PTSD.
160. A pharmaceutical composition comprising cyclobenzaprine, amitriptyline or pharmaceutically acceptable salts thereof for the treatment or prevention of PTSD, ASD or one or more associated symptoms thereof in a subject in need thereof, wherein the treatment comprises:
   a) administering the medicament daily to the subject;
   b) assessing the efficacy of the treatment periodically over a course of the treatment;
   c) suspending the administration of the medicament when the efficacy diminishes;
   d) resuming the administration of the medicament 4 weeks after suspending the treatment;
   wherein steps (a)-(d) may be repeated one or more times.
161. A pharmaceutical composition comprising cyclobenzaprine, amitriptyline or pharmaceutically acceptable salts thereof for the treatment or prevention of PTSD, ASD or one or more associated symptoms thereof in a subject in need thereof, wherein the treatment comprises:
   a) administering the medicament daily to the subject;
   b) suspending the administration after about 4 weeks;
   c) resuming the administration about 4 weeks after suspending the administration;
   wherein steps (a)-(c) may be repeated one or more times.
162. The pharmaceutical composition of embodiment 160 or 161, wherein the treatment or prevention is of PTSD, and the subject has experienced a traumatic event less than or equal to about 9 years prior to the commencement of treatment.
163. The pharmaceutical composition of embodiment 162, wherein the efficacy of the treatment is measured at least about every 2 weeks after the treatment begins.
164. The pharmaceutical composition of embodiment 163, wherein the efficacy of the treatment is assessed based on the subject's Clinician Administered PTSD Scale for DSM-5 (CAPS-5) score.
165. The pharmaceutical composition of any one of embodiments 160-164, wherein the cyclobenzaprine or amitriptyline in the pharmaceutical composition is the cyclobenzaprine or amitrptyline free base.
166. The pharmaceutical composition of any one of embodiments 160-164, wherein the cyclobenzaprine or amitriptyline in the pharmaceutical composition is a pharmaceutically acceptable cyclobenzaprine or amitriptyline salt.
167. The pharmaceutical composition of any one of embodiments 160-166, wherein the medicament is administered sublingually, buccally, orally, in a suppository, intravenously, intramuscularly, subcutaneously, inhalationally, intranasally, in a thin film, transdermally, parenterally, rectally, or vaginally.
168. The pharmaceutical composition of embodiment 167, wherein the medicament is administered sublingually.
169. The pharmaceutical composition of any one of embodiments 160-168, wherein the pharmaceutical composition comprises a basifying agent.
170. The pharmaceutical composition of embodiment 169, wherein the basifying agent is selected from the group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate.
171. The pharmaceutical composition of any one of embodiments 160-170, wherein the efficacy of the treatment increases with decreasing time between the commencement of treatment and the traumatic event.
172. The pharmaceutical composition of any one of embodiments 160-171, wherein the amount of cyclobenzaprine, amitriptyline or pharmaceutically acceptable salts thereof that is in the medicament is between about 0.1 mg and about 50 mg/day.
173. The pharmaceutical composition of embodiment 172, wherein the amount of cyclobenzaprine or pharmaceutically acceptable salt thereof that is in the medicament is between about 0.5 mg and about 30 mg/day.
174. The pharmaceutical composition of embodiment 173, where the amount of cyclobenzaprine, amitriptyline or pharmaceutically acceptable salts thereof that is in the medicament is between about 1 mg and about 20 mg/day.
175. The pharmaceutical composition of embodiment 160-171, wherein the amount of amitriptyline or pharmaceutically acceptable salt thereof that is in the medicament is between about 0.1 mg and about 150 mg/day.
176. The pharmaceutical composition of embodiment 175, wherein the amount of amitriptyline or pharmaceutically acceptable salt thereof that is in the medicament is between about 1.0 mg and about 90 mg/day.
177. The pharmaceutical composition of embodiment 176, where the amount of amitriptyline or pharmaceutically acceptable salt thereof that is in the medicament is between about 3 mg and about 60 mg/day.
178. The pharmaceutical composition of embodiment 160 or 161, wherein the pharmaceutical composition is for sequential or concurrent administration in combination with a compound selected from the group consisting of an alpha-1-adrenergic receptor antagonist, a beta-adrenergic antagonist, an anticonvulsant, a selective serotonin reuptake inhibitor and a serotonin-norepinephrine reuptake inhibitor.
179. The pharmaceutical composition of embodiment 178, wherein the alpha-1-adrenergic receptor antagonist is prazosin.
180. The pharmaceutical composition of embodiment 178, wherein the selective serotonin reuptake inhibitor is sertraline, paroxetine, fluoxetine, citalopram or escitalopram.
181. The pharmaceutical composition of embodiment 160 or 161, wherein the pharmaceutical composition is administered in combination with psychotherapeutic intervention during the course of treatment.
182. The pharmaceutical composition of any one of embodiments 160-181, wherein the treatment or prevention is of PTSD, and at least one of the symptoms of PTSD are eliminated or ameliorated.
183. The pharmaceutical composition of embodiment 182 wherein the symptoms of PTSD are selected from the group consisting of intrusion symptoms, avoidance symptoms, cognition and mood symptoms, arousal and reactivity symptoms, difficulty falling sleep, irritability, difficulty concentrating, hypervigilance, and persistent exaggerated startle response.
184. The pharmaceutical composition of embodiment 160 or 161, wherein the subject has experienced a criterion A trauma.
185. The pharmaceutical composition of embodiment 184, wherein the criterion A trauma results in ASD or symptoms thereof.
186. The pharmaceutical composition of embodiment 185, wherein at least one of the symptoms of ASD are eliminated or ameliorated.
187. The pharmaceutical composition of embodiment 186, wherein the symptoms of ASD are selected from the group consisting of reexperiencing symptoms, avoidance symptoms, arousal symptoms, difficulty with sleep, nightmares, irritability, difficulty concentrating, hypervigilance, persistent exaggerated startle response, feelings such as not knowing where you are, and feeling as if you are outside of your body.

### DESCRIPTION OF THE DRAWINGS

FIG. 1 depicts the least squares mean change in CAPS-5 scores against years since trauma after 4 weeks of treatment and after 12 weeks of treatment with 5.6 mg sublingual cyclobenzaprine (TNX-102 SL).

FIG. 2 depicts the least squares mean change in CAPS-5 scores from the baseline against years since trauma after 4 weeks of treatment with either the placebo (PBO) or 5.6 mg sublingual cyclobenzaprine (TNX).

FIG. 3 depicts the least squares mean change in CAPS-5 scores from the baseline against years since trauma after 12 weeks of treatment with either the placebo (PBO) or 5.6 mg sublingual cyclobenzaprine (TNX).

FIG. 4 is a scatter plot depicting the change in CAPS-5 scores from the baseline against time in months since trauma after 4 weeks of treatment with either the placebo or 5.6 mg sublingual cyclobenzaprine (TNX-102 SL).

FIG. 5 depicts six boxplots showing the change in CAPS-5 scores from the baseline against time since trauma after 4, 8 or 12 weeks of treatment, relative to placebo, where a diminished response to the treatment with cyclobenzaprine (TNX-102 SL) is seen in subjects with a history of smoking (Y, bottom) than in subjects without a history of smoking (N, top).

FIG. 6 is a chart depicting the average CAPS-5 baseline scores, and the CAPS-5 scores for subjects who have received treatment with cyclobenzaprine (TNX-102 SL) for 4 weeks and who have experienced a traumatic event less than or equal to 109 months (˜9 years) prior to commencement of treatment.

FIG. 7 is a chart depicting the CAPS-5 scores for subjects who have received treatment with 5.6 mg sublingual cyclobenzaprine (TNX-102 SL) for 8 weeks, and who have experienced a traumatic event less than or equal to 109 months (˜9 years) prior to commencement of treatment.

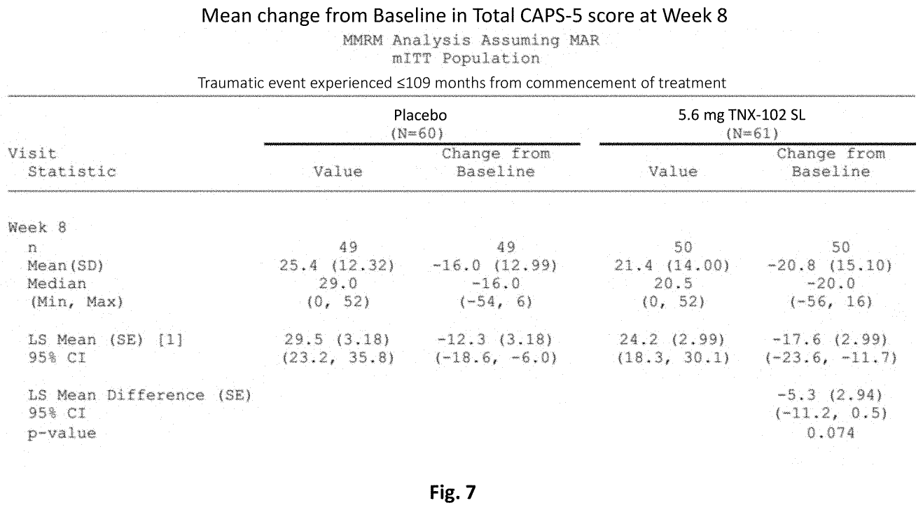

FIG. 8 is a chart depicting the CAPS-5 scores for subjects who have received treatment with 5.6 mg sublingual cyclobenzaprine (TNX-102 SL) for 12 weeks, and who have experienced a traumatic event less than or equal to 109 months (˜9 years) prior to commencement of treatment.

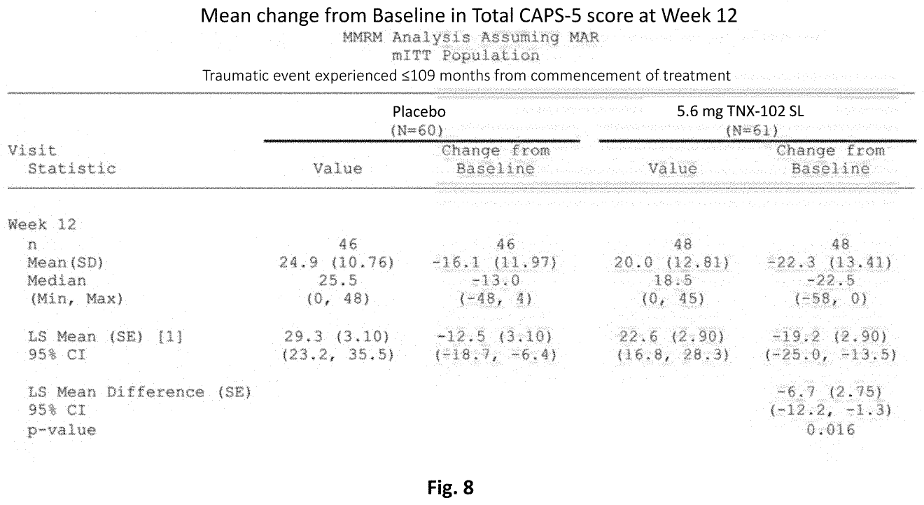

FIG. 9 is a chart depicting the average CAPS-5 baseline scores, and the CAPS-5 scores for subjects who have received treatment with 5.6 mg sublingual cyclobenzaprine (TNX-102 SL) for 4 weeks and who have experienced a traumatic event more than 109 months (˜9 years) prior to commencement of treatment.

FIG. 10 is a chart depicting the CAPS-5 scores for subjects who have received treatment with 5.6 mg sublingual cyclobenzaprine (TNX-102 SL) for 8 weeks, and who have experienced a traumatic event more than 109 months (˜9 years) prior to commencement of treatment.

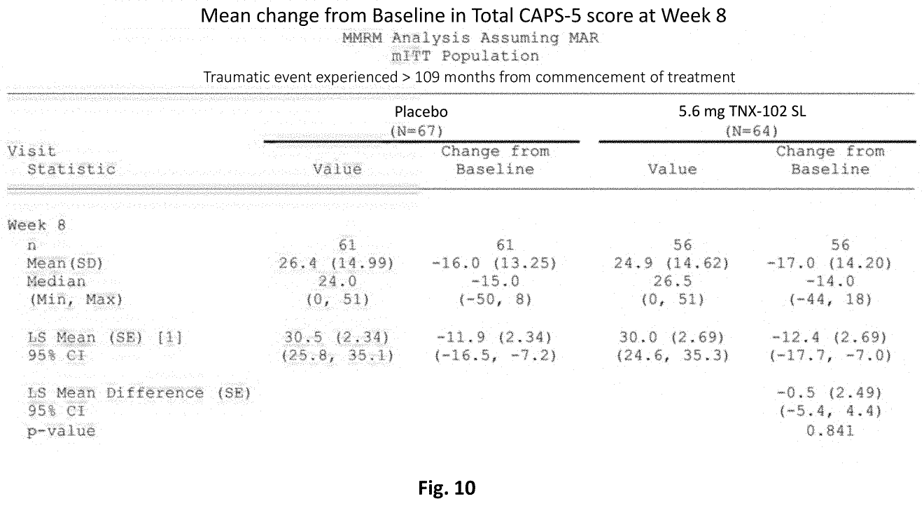

FIG. 11 is a chart depicting the CAPS-5 scores for subjects who have received treatment with cyclobenzaprine (TNX-102 SL) for 12 weeks, and who have experienced a traumatic event more than 109 months (˜9 years) prior to commencement of treatment.

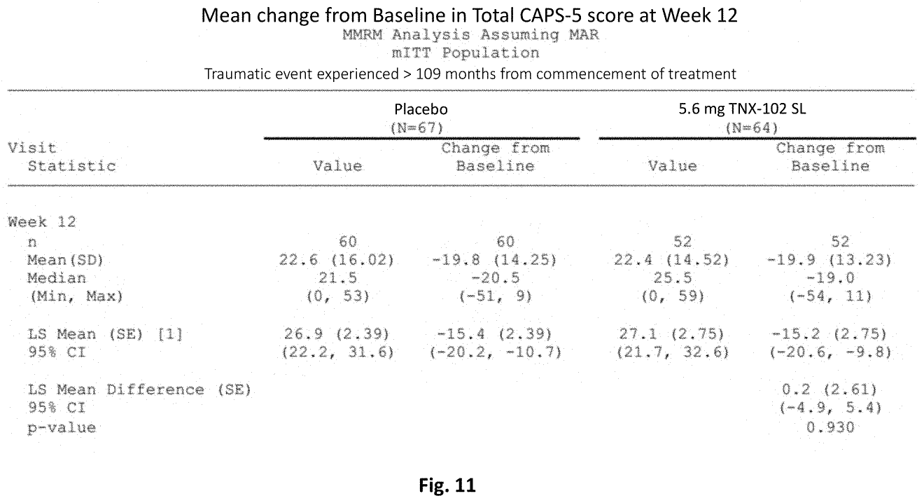

FIG. 12 is a chart depicting the remission rates between subjects who experienced an adverse event (ON/OT/NT+) from 5.6 mg sublingual cyclobenzaprine administration (TNX 5.6 mg) and subjects who did not experience an adverse event (ON/OT/NT−) from 5.6 mg sublingual cyclobenzaprine administration. The remission rates were similar between both groups suggesting that the occurrence of an adverse event did not unblind the study.

FIG. 13 is a chart depicting the least square mean change in from baseline in CAPS-5 derealization scores for subjects who received placebo, or sublingual cyclobenzaprine (TNX-102 SL 5.6 mg, and TNX-102 SL 2.8 mg) over the course of 12 weeks of treatment.

FIG. 14 depicts the treatment responsiveness over the course of PTSD. Panel a depicts the time frames in which clinical trials with sublingual cyclobenzaprine (P201 AtEase trial and P301 HONOR trial) were conducted. Panel b depicts the time frames in which selected clinical trials with various drugs were conducted in civilian vs military subjects with PTSD over the course of the disease beginning at trauma (time 0). Panel c depicts the survival curve showing the proportion surviving without recovery versus the time since trauma. Panel d depicts the progression of the disease from the rapid recovery phase (ASD) to the remitting phase and finally the persistent phase.

FIG. 15 depicts the rates of remission for subjects who experienced a traumatic event less than or equal to 9 years before receiving treatment with TNX 5.6 mg in the P301 trial (right), and those with a CAPS-5 greater than or equal to 33 in the P201 trial (left). Similar rates of remission were observed in both trials.

### DETAILED DESCRIPTION

### Definitions and General Techniques

Unless otherwise defined herein, scientific and technical terms used in this application shall have the meanings that are commonly understood by those of ordinary skill in the art. In case of conflict, the present specification, including definitions, will control.

Throughout this specification and embodiments, the word “comprise,” or variations such as “comprises” or “comprising,” will be understood to imply the inclusion of a stated integer or group of integers but not the exclusion of any other integer or group of integers.

The term “including” or “includes” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a”, “an” and “the” are used herein to refer to one or to more than one (i.e., to at least one) of the grammatical object of the article.

Notwithstanding that the disclosed numerical ranges and parameters are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments are described in terms of a Markush group or other grouping of alternatives, the present application encompasses not only the entire group listed as a whole, but each member of the group individually and all possible subgroups of the main group, and also the main group absent one or more of the group members. The present application also envisages the explicit exclusion of one or more of any of the group members in the embodimented disclosure.

Exemplary methods and materials are described herein, although methods and materials similar or equivalent to those described herein can also be used in the practice or testing of the various aspects and embodiments. The materials, methods, and examples are illustrative only and not intended to be limiting.

In order for the disclosure to be more readily understood, certain terms are first defined. These definitions should be read in light of the remainder of the disclosure as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “about” refers to a value or parameter that includes (and describes) embodiments that are directed to that value or parameter per se. For example, description referring to “about X” includes description of “X”. Numeric ranges are inclusive of the numbers defining the range. Unless specified otherwise, the term “about” when used in the context of a dosage of a compound to be administered to a patient, permits a variation of ±10% of a given value or range. As used herein, the term “about” when used in the context of years since a subject suffering from PTSD has experienced traumatic event permits a variation of ±6 months. As used herein, the term “about” when used in the context of months since a subject suffering from ASD has experienced a traumatic event permits a variation of ±1 week. As used herein, the term “about” when used in the context of administration periods and suspension periods of treatment permits a variation of ±5 days.

As used herein, the term “treat” and its cognates refers to taking steps to obtain beneficial or desired results, i.e. to obtain a full or partial amelioration of at least one of the symptoms associated with PTSD or ASD, preferably remission of PTSD or ASD. Methods to measure the full or partial improvement or amelioration of PTSD or ASD symptoms are known by the skilled in the art and include the Clinician-Administered PTSD Scale for DSM-5 (CAPS-5), the Clinician Global Impression-Improvement (CGI-I) scale, the Sheehan Disability Scale (SDS), the Patient Global Impression of Change scale (PGIC), the Beck Depression Inventory-II scale, the Davidson Trauma Scale, the Dissociative Experiences Scale, and the PTSD Check List (PCL). An improved score using these methods is indicative of successful “treatment”. As used in the present disclosure, the CAPS-5 method is a 30-item structured interview that is used to assess PTSD or ASD symptoms. The first 20 questions target the symptoms of PTSD as defined in DSM-5, and some of the remaining items target the onset, duration, and impact of symptoms on the social and occupational functioning of the subject. The final two items (items 29 and 30) focus on derealization symptoms and depersonalization symptoms to allow subtyping of PTSD, the ‘dissociative’ subtype if either or both is present at a clinically significant level. These two symptoms also are among the nine or more required for a diagnosis of ASD. A decrease of about 5±3 points in the subject's CAPS-5 score is indicative of a successful “treatment.”

A “patient”, “subject”, or “individual” are used interchangeably and preferably refer to a human being.

“Administering” or “administration of” a substance, a compound or an agent to a subject can be carried out using one of a variety of methods known to those skilled in the art. For example, a compound or an agent can be administered sublingually, buccally, orally, in a suppository, intravenously, intramuscularly, subcutaneously, inhalationally, intranasally, in a thin film, transdermally, parenterally, rectally, or vaginally. The administration can also be performed, for example, once, or a plurality of times per day, and/or over one or more longer periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

As used herein, “administering daily” refers to the administration of a pharmaceutical composition according to any one of the administration methods stated above once or multiple times daily. For example, 5 mg/day can be administered in one dose or in several doses totaling 5 mg. One dose is preferred.

As used herein, the terms “prevent”, “preventing” and “prevention” refer to the elimination of the recurrence or onset of, or a reduction in one or more symptoms of a disorder in a subject as a result of the administration of a therapy (e.g., a therapeutic agent).

As used herein, the term “post-traumatic stress disorder” or PTSD refers to a disorder that develops after exposure to a traumatic event, including a criterion A traumatic event and is characterized by symptoms including, but not limited to, difficulty with sleep, nightmares, irritability, difficulty concentrating, hypervigilance, and a persistent exaggerated startle response. Those suffering from PTSD also have at least one intrusion symptom, at least one avoidance symptom, at least two cognition and mood symptoms, and at least two arousal and reactivity symptoms. Intrusion symptoms include flashbacks, bad dreams, and frightening thoughts. Avoidance symptoms include staying away from places, events or objects that are reminders of the experience, and avoiding thoughts or feelings related to the traumatic event. Arousal and reactivity symptoms include exaggerated startled response, feelings of tension, difficulty sleeping, and irritability. Cognition and mood symptoms include trouble remembering key features of the traumatic event, negative thoughts about oneself or the world, distorted feelings like guilt or blame, and loss of interest in enjoyable activities. PTSD can be subtyped further as dissociative PTSD. This subtype is characterized by symptoms such as depersonalization and derealization. Depersonalization symptoms consist of feelings as if oneself is not real, and derealization symptoms consist of feelings as if the world is not real.

As used herein, the term “acute stress disorder” or ASD refers to a disorder that develops after exposure to a traumatic event, including a criterion A traumatic event and is characterized by severe anxiety, dissociation, reexperiencing the traumatic event, avoidance, and distress. ASD is associated with many of the same symptoms as PTSD, however ASD lasts from about 2 days to about one month and generally occurs within about one month of the traumatic event. The subject must also have at least one reexperiencing symptom, at least one avoidance symptom, and at least one arousal symptom to be diagnosed with ASD. If symptoms persist longer than about one month, the disorder has evolved into PTSD. Additionally, further derealization and depersonalization symptoms such as feelings such as not knowing where you are or feeling as if you are outside of your body are more likely to be associated with ASD than with PTSD. While ASD is not necessarily a predictor for the development of PTSD, those who are diagnosed with ASD frequently develop PTSD.

As used herein, the term “cyclobenzaprine” includes deuterated cyclobenzaprine and any pharmaceutically acceptable salts thereof, wherein either one or both of the amino-methyl groups are deuterated partially or completely (e.g., 3-(5H-Dibenzo[a,d]cyclohepten-5-ylidene)-N,N-di(methyl-d3)-1-propanamine or 3-(5H-Dibenzo[a,d]cyclohepten-5-ylidene)-N-methyl-N-(methyl-d3)-1-propanamine, and their pharmaceutically acceptable salts). The term “cyclobenzaprine” also includes eutectics of cyclobenzaprine HCl and mannitol, wherein the eutectic ratio is either 75%±2% cyclobenzaprine HCl by weight and 25%±2% β-mannitol by weight, or 65%±2% cyclobenzaprine HCl by weight and 35%±2% δ-mannitol by weight. Exemplary eutectic compositions can be found in U.S. Pat. Nos. 9,636,408, 9,956,188 and U.S. patent application Ser. Nos. 15/941,484, and 14/776,624 and 15/511,287 which are hereby incorporated by reference in their entirety.

As used herein, the term “amitriptyline” includes deuterated amitriptyline and any pharmaceutically acceptable salts thereof, wherein either one or both of the amino-methyl groups are deuterated partially or completely (e.g., 3-(10, 11-dihydro-5H-dibenzo [a,d] cycloheptene-5-ylidene)-N,N-di(methyl-d3)-1-propanamine, or 3-(10, 11-dihydro-5H-dibenzo [a,d] cycloheptene-5-ylidene)-N-methyl-N-(methyl-d3)-1-propanamine, and their pharmaceutically acceptable salts). The term “amitriptyline” also includes eutectics of amitriptyline HCl and mannitol, wherein the eutectic ratio is 75%±2% amitriptyline HCl by weight and 25%±2% β-mannitol by weight. Exemplary eutectic compositions can be found in U.S. patent application Ser. Nos. 15/941,484, and 14/776,624, which are incorporated by reference in their entirety.

As used herein, the term “therapeutically effective amount” of cyclobenzaprine, amitriptyline or pharmaceutically acceptable salts thereof refers to the amount of the compound that treats or prevents or eliminates or alleviates with at least one of the symptoms associated with PTSD or ASD. A physician can readily determine when symptoms are prevented or alleviated or eliminated, for example through clinical observation of a subject, or through reporting of symptoms by the subject or its caregiver during the course of treatment. One skilled in the art can readily determine the amount of a cyclobenzaprine, amitriptyline or pharmaceutically acceptable salts thereof to be administered, by taking into account factors such as the size, weight, age and sex of the subject, the extent of disease penetration or persistence and severity of symptoms, and the route of administration.

As used herein, the term “traumatic event”, as the causative factor of PTSD or ASD refers to a direct or indirect personal experience that causes physical, emotional, spiritual or psychological harm. Traumatic events may preferentially include criterion A traumatic events which involve actual or threatened death or serious injury, or other threat to a subject's physical integrity; or witnessing an event that involves death, injury, or a threat to the physical integrity of another person; or learning about unexpected or violent death, serious harm, or threat of death or injury experienced by a family member or other close associate. Examples of traumatic events that are experienced directly include, but are not limited to, military combat, violent personal assault, being kidnapped, being taken hostage, terrorist attack, torture, incarceration as a prisoner of war or in a concentration camp, natural or manmade disasters, severe automobile accidents, or being diagnosed with a life-threatening illness. For children, sexually traumatic events may include developmentally inappropriate sexual experiences without threatened or actual violence or injury. Witnessed, or indirect, events include, but are not limited to, observing the serious injury or unnatural death of another person due to violent assault, accident, war or disaster or unexpectedly witnessing a dead body or body parts. Events experienced by another that are learned about include, but are not limited to, violent personal assault, serious accident, or serious injury experienced by a family member or a close friend; learning about the sudden, unexpected death of a family member or a close friend; or learning that one's child has a life-threatening disease or through exposure to aversive details of trauma usually through the course of professional duties, e.g., first responders or medics. The disorder may be especially severe or long lasting when the stressor is of human design (e.g., torture, rape). Shortly after the trauma occurs, people may develop symptoms such as nightmares, intrusive memories, exaggerated startle response, feelings such as not knowing where you are, or feelings as if you are outside of your body. If these symptoms are of sufficient severity, the syndrome is called acute stress disorder (ASD). If the symptoms persist for about 4 weeks, the disorder may evolve into PTSD. Traumatic events may also include exposure to divorce, abandonment, and imprisonment.

### Method for Treating or Preventing PTSD and Associated Symptoms and Method for Treating ASD and Associated Symptoms

In one aspect, the disclosure relates to a method for treating post-traumatic stress disorder (PTSD) or one or more symptoms thereof in a subject who has experienced a PTSD-causing traumatic event less than or equal to about 9 years prior to the commencement of treatment comprising, administering to the subject a pharmaceutical composition comprising a therapeutically effective amount of cyclobenzaprine, amitriptyline, or pharmaceutically acceptable salts thereof.

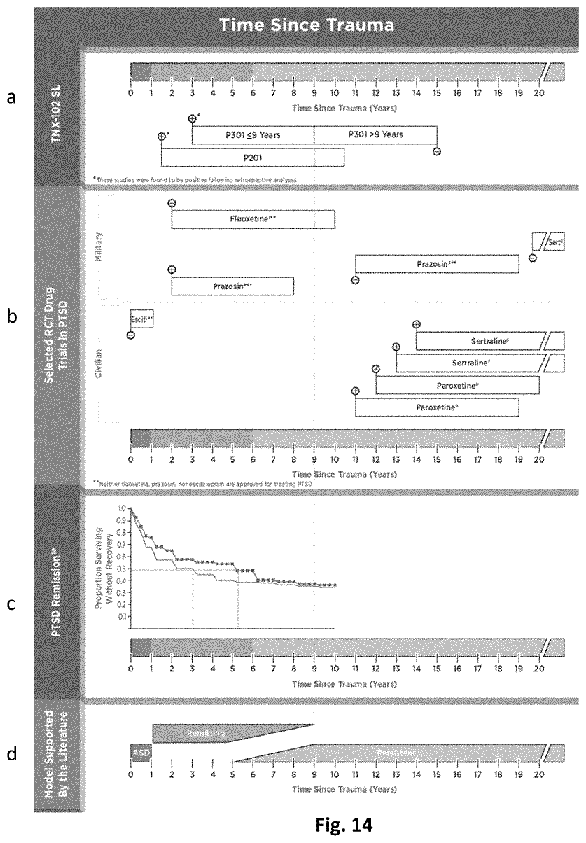

PTSD is associated with three separate phases; a rapid recovery phase, a remitting phase, and a persistent phase (FIG. 14). Without wishing to be bound by theory, the extent to which subjects respond to a PTSD treatment may depend on which phase of PTSD the subject is in. The rapid recovery phase corresponds to the first year after the onset of symptoms following a PTSD-causing traumatic event and represents the period of time during which treatment may be most effective. Survival curves plotting the proportion of subjects surviving without recovery versus the time since trauma indicate that the largest percentage of those who achieve remission of PTSD, do so within the first year (Kessler, 1995). Following the rapid recovery phase, the survival curve decreases at a more gradual rate for about 5 years to about 9 years after the onset of symptoms. This period is identified as the remitting phase. After about 9 years, the survival curve levels off, and represents the phase in which remission of PTSD is the most difficult to achieve. In some embodiments, the administration of the pharmaceutical composition of this disclosure within the rapid recovery phase of PTSD is more effective than the administration of a treatment within the remitting phase. In other embodiments, the administration of the pharmaceutical composition of this disclosure within the remitting phase of PTSD is more effective than the administration within the persistent phase. In some embodiments, the treatment of PTSD according to this disclosure is performed in subjects which are on the rapid recovery phase of PTSD. In other embodiments, the treatment of PTSD according to this disclosure is performed in subjects who are in the remitting phase of PTSD. Optionally, the treatment of PTSD according to this disclosure is performed on subjects which are in the persistent phase of PTSD. In certain embodiments, the method for treatment of PTSD comprises administering a pharmaceutical composition comprising cyclobenzaprine, amitriptyline or pharmaceutically acceptable salts thereof to subjects who have experienced a PTSD-causing traumatic event within 9 years or less prior to the commencement of treatment. As the time between the traumatic event and the commencement of treatment decreases, the efficacy of the treatment will increase. In some aspects of this disclosure, the treatment is administered to a patient within 1 month, 2 months, 3 months, 4 months, 5 months, 6 months, 1 year, 2 years, 3 years, 4 years, 5 years, 6 years, 7 years, 8 years, 9 years or 9.5 years from the traumatic event.

In some aspects of this disclosure, the development of PTSD is prevented by treating ASD. The initiation of ASD symptoms generally occur immediately (for example within 30 minutes or up to a few days or weeks) following ASD-causing traumatic event. The symptoms generally then become increasingly severe. If the severity of the symptoms persists for more than about 4 weeks, the subject may be diagnosed with PTSD. ASD shares many of the same symptoms as PTSD including emotional numbness, restlessness, anxiety, irritability, issues concentrating, flashbacks and sleep disturbances. However, ASD is generally more associated with dissociative symptoms, such as emotional disconnection, difficulty experiencing pleasure, temporary amnesia, depersonalization and derealization. It is thought that these dissociative symptoms may play a role in preventing the subject from processing the traumatic event fully, and may hinder the subject's recovery process. Without wishing to be bound by theory, interventions as early as possible of the ASD-causing traumatic event may prevent some patients suffering from ASD from developing into full blown PTSD.

In certain aspects of this disclosure, method of preventing the development of PTSD in patients suffering from ASD is provided. The development of PTSD can be prevented by treating a subject in need thereof soon after they have experienced a PTSD-causing or ASD-causing traumatic event. By decreasing the amount of time between the traumatic event and the commencement of treatment, the efficacy of the treatment can be enhanced. In some aspects of this disclosure, the treatment is commenced within 4 weeks of the traumatic event, preferably within the same day as the traumatic event, 1 day, 1 week, 2 weeks, 3 weeks or 4 weeks of the traumatic event. In certain aspects, this “immediate” treatment prevents the development of PTSD or ASD for those who have experienced a PTSD-causing or ASD-causing traumatic event. In some aspects of this disclosure, the traumatic event can be classified as a criterion A traumatic event

In another aspect, the disclosure relates to a method for treating acute stress disorder (ASD) or one or more symptoms thereof, in a subject who has experienced an ASD-causing traumatic event, including a criterion A traumatic event, comprising, administering to the subject, a pharmaceutical composition comprising a therapeutically effective amount of cyclobenzaprine, amitriptyline or pharmaceutically acceptable salts thereof, wherein the subject experienced the traumatic event less than or equal to 1 month ±5 days prior to the commencement of treatment.

In some aspects, the pharmaceutical composition of the disclosure is formulated for sublingual, buccal, oral, suppository, intravenous, intramuscular, subcutaneous, inhalational, intranasal, thin film, transdermal, parenteral, rectal, or vaginal administration. In some aspects, the pharmaceutical composition is administered in combination (sequentially or concurrently) with psychotherapeutic therapies or environmental intervention. Psychotherapeutic therapies include but are not limited to exposure therapies, eye movement desensitization and reprocessing therapy, somatic therapies, cognitive behavioral therapy, and ecotherapy.

In some embodiments, the pharmaceutical composition comprising pharmaceutically acceptable salts of cyclobenzaprine or amitriptyline further comprises a basifying agent. As used herein, a “basifying agent” refers to an agent or a substance that increases the local pH of the liquid near a mucosal surface. Examples of basifying agents which can be used in the present disclosure include, but are not limited to, potassium dihydrogen phosphate (monophosphate, monobasic potassium phosphate, KH₂PO₄), dipotassium hydrogen phosphate (dipotassium phosphate, dibasic potassium phosphate, K₂HPO₄), tripotassium phosphate (K₃PO₄), sodium dihydrogen phosphate (monosodium phosphate, monobasic sodium phosphate, NaH₂PO₄), disodium hydrogen phosphate (disodium phosphate, dibasic sodium phosphate, Na₂HPO₄), trisodium phosphate (Na₃PO₄), bicarbonate or carbonate salts, dipotassium citrate, tripotassium citrate, TRIS buffer, potassium acetate, sodium acetate, disodium citrate, trisodium citrate, borate, hydroxide, silicate, nitrate, dissolved ammonia, the conjugate bases of some organic acids (including bicarbonate and sulfide) that raises the pH of a solution containing a compound (e.g., cyclobenzaprine or a pharmaceutically acceptable salt thereof) useful in the compositions and methods of the invention.

In some aspects of this disclosure, the pharmaceutical composition comprises a eutectic comprising pharmaceutically acceptable salts of cyclobenzaprine or amitriptyline and mannitol. A eutectic is a mixture of chemical compounds or elements that has a single chemical composition that melts at a lower temperature than any other composition made up of the same ingredients. A composition comprising a eutectic is known as the eutectic composition and its melting temperature is known as the eutectic temperature.

In some embodiments, the method of the present disclosure involves administering pharmaceutical compositions comprising cyclobenzaprine or pharmaceutically acceptable salt thereof to a subject in need. In some embodiments, a therapeutically effective amount of cyclobenzaprine or pharmaceutically acceptable salts thereof administered to a subject is between about 0.1 mg to about 30 mg/day, between about 1 to about 20 mg/day, less than about 10 mg/day, less than about 5 mg/day, about 5.6 mg/day, or about 2.8 mg/day. Higher or lower doses are also contemplated. In certain embodiments, the amount of cyclobenzaprine or pharmaceutically acceptable salt thereof administered to a subject is between about 0.1 mg and about 50 mg/day. In some embodiments, the amount of cyclobenzaprine or pharmaceutically acceptable salts thereof administered to a subject is between about 0.5 and about 30 mg/day. In some embodiments, the amount of cyclobenzaprine or pharmaceutically acceptable salts thereof administered to a subject is between about 1 mg and about 20 mg/day.

In some aspects, the methods of the disclosure involve administering pharmaceutical compositions comprising amitriptyline or pharmaceutically acceptable salts thereof to a subject in need. In some embodiments, a therapeutically effective amount of amitriptyline or pharmaceutically acceptable salt thereof administered to a subject is between about 0.1 mg to about 90 mg/day, between about 1 to about 60 mg/day, less than about 30 mg/day, or less than about 15 mg/day. Higher or lower doses are also contemplated. In certain embodiments, the amount of amitriptyline or pharmaceutically acceptable salt thereof administered to a subject is between about 0.1 mg and about 150 mg/day. In some embodiments, the amount of amitriptyline or pharmaceutically acceptable salt thereof administered to a subject is between about 0.5 and about 30 mg/day. In some embodiments, the amount of amitriptyline or pharmaceutically acceptable salt thereof administered to a subject is between about 1 mg and about 60 mg/day.

In some aspects of this disclosure, the cyclobenzaprine, amitriptyline or pharmaceutically acceptable salts thereof is administered in combination with one or more agents which may further alleviate the symptoms of PTSD or ASD. These agents may be administered sequentially or concurrently with cyclobenzaprine, amitriptyline or pharmaceutically acceptable salts thereof. Examples of agents which can be administered with the cyclobenzaprine, amitriptyline or pharmaceutically acceptable salts thereof include, but are not limited to, an alpha-1-adrenergic receptor agonist, a beta-adrenergic antagonist, an anticonvulsant, a selective serotonin reuptake inhibitor or a serotonin-norepinephrine reuptake inhibitor. Exemplary selective serotonin reuptake inhibitors or serotonin-norepinephrine reuptake inhibitors include, but are not limited to, bupropion, citalopram, desvenlafaxine, duloxetine, escitalopram, fluoxetine, fluvoxamine, milnacipran, paroxetine, sertraline, trazodone, and venlafaxine. Exemplary anticonvulsants include, but are not limited to, carbamazepine, gabapentin, lamotrigine, oxcarbazepine, pregabalin, tiagabine, topiramate, and valproate. Exemplary alpha-1-adrenergic receptor antagonists include, but are not limited to, prazosin.

In some embodiments, in preparing pharmaceutical compositions of this disclosure for sublingual administration, the cyclobenzaprine, amitriptyline of pharmaceutically acceptable salts thereof can be combined with one or more solid or liquid inactive ingredients to form tablets, capsules, pills, powders, granules, sprays or other suitable sublingual dosage forms. For example, in some aspects, the cyclobenzaprine, amitriptyline or pharmaceutically acceptable salts thereof, can be combined with at least one pharmaceutically acceptable carrier, such as a solvent, filler, binder, humectant, disintegrating agent, solution retarder, absorption accelerator, wetting agent absorbent or lubricating agent. In other aspects, the cyclobenzaprine, amitriptyline or pharmaceutically acceptable salts thereof are combined with carboxymethylcellulose calcium, magnesium stearate, mannitol or starch, and is formed into tablets by conventional tableting methods. Pharmaceutical compositions suitable for use in the present application are described, for example, in WO2013188847, which is hereby incorporated by reference into the specification.

In one aspect, the disclosure relates to a method of treating or preventing PTSD or ASD and associated symptoms in a subject in need thereof, comprising:
a) administering daily to the subject a pharmaceutical composition comprising cyclobenzaprine, amitriptyline or pharmaceutically acceptable salts thereof;
b) assessing the efficacy of the treatment periodically over a course of the treatment;
c) suspending the treatment when the efficacy diminishes;
d) resuming the treatment 4 weeks after suspending the treatment;
   wherein steps (a)-(d) may be repeated one or more times.

In certain aspects of this disclosure, the pharmaceutical composition is administered to a subject based on an intermittent administration schedule. The pharmaceutical composition may be administered daily for a first administration period of about 4±2 weeks, which is followed by a second suspension period of about 4±2 weeks in which the patient does not receive treatment. The administration period and the suspension period can be repeated one or more times. In other aspects, the pharmaceutical composition is administered to a subject without the suspension period. The intermittent administration of the pharmaceutical composition can be beneficial for subjects who experience a decrease in efficacy of the treatment after an extended period of time.

In some aspects of this disclosure, the efficacy of the treatment disclosed is assessed based on a subject's Clinician-Administered PTSD scale for DSM-5 (CAPS-5) score relative to the subject's baseline state at the beginning of treatment. The symptoms are assigned a severity rating ranging from absent (0) to extreme (4). The scores for each symptom are added up resulting in an overall CAPS-5 score. A decrease in the subject's CAPS-5 score during the course of treatment indicates that the treatment is effective. In some embodiments, the efficacy of the treatment is measured 1 week, 2 weeks, 3 weeks, 4 weeks, 5 weeks, 6 weeks, 7 weeks, 8 weeks, 9 weeks, 10 weeks, 11 weeks and/or 12 weeks after the commencement of the treatment. A decrease of about 5±3 points from the subject's baseline score is indicative of an effective treatment. Alternatively, the efficacy of the treatment can be measured using other scales or scores common in the art for determining the severity of PTSD or ASD. These scales include, but are not limited to, the Clinician Global Impression-Improvement (CGI-I) scale, the Sheehan Disability Scale (SDS), the Patient Global Impression of Change scale (PGIC), the Beck Depression Inventory-II scale, the Davidson Trauma Scale, the Dissociative Experiences Scale, and the PTSD Check List (PCL). These scales should be used in comparison to the subject's baseline state at the beginning of treatment and after the treatment commences. An improved score indicates that the treatment is effective.

In some embodiments, the efficacy of the treatment may be used to determine an intermittent dosing schedule for a subject. In some embodiments, the method of treating or preventing the development of PTSD or ASD in a subject in need thereof comprises monitoring the efficacy of the treatment periodically over the course of the treatment to determine a point of suspension (i.e., a decrease in efficacy) in the administration schedule. The efficacy can be measured weekly, every other week, or monthly during a period of time wherein the subject is administered the pharmaceutical composition once daily. If the efficacy of the treatment diminishes, the treatment is suspended for about 4±2 weeks, and then resumed for a period of time corresponding with the period of time over which treatment has been determined to be effective, or preferably by monitoring efficacy as before.

### Method of Determining the Therapeutic Dosage for the Treatment of PTSD

In one aspect, the disclosure relates to method of determining a therapeutic dosage of cyclobenzaprine or pharmaceutically acceptable salts thereof for the treatment of PTSD or ASD comprising:
a) obtaining a suitable cell or tissue sample from a subject suffering from PTSD;
b) identifying the CYP1A2, CYP2D6, and CYP3A4 genotype of said subject to determine if the patient has a high cyclobenzaprine metabolizer genotype;
c) assessing the subjects medical history for a history of smoking;
   wherein if the subject has at least one of the criteria identified in step (b) or (c), the dose of cyclobenzaprine administered to the subject is greater than about 5 mg/day;
   wherein if the subject does not have at least one of the criteria identified in step (b) or (c, the dose of cyclobenzaprine administered to the subject is about 5.6 mg/day or less.

In another aspect, the disclosure relates to method of determining a therapeutic dosage of amitriptyline or pharmaceutically acceptable salts thereof for the treatment of PTSD or ASD comprising:
a) obtaining a suitable cell or tissue sample from a subject suffering from PTSD;
b) identifying the CYP1A2, CYP2D6, and CYP3A4 genotype of said subject to determine if the patient has a high amitriptyline metabolizer genotype;
c) assessing the subjects medical history for a history of smoking;
   wherein if the subject has at least one of the criteria identified in step (b) or (c), the dose of amitriptyline administered to the subject is greater than 11 mg/day;
   wherein if the subject does not have at least one of the criteria identified in step (b) or (c, the dose of amitriptyline administered to the subject is 11.2 mg/day or less.

In some aspects of this disclosure, a pharmacogenomic test to identify cytochrome CYP1A2, CYP2D6 and CYP3A4 genotypes may be used to predict the metabolism of cyclobenzaprine or amitriptyline by certain subjects in order to select an effective dose of cyclobenzaprine or amitriptyline to administer. The presence of different alleles of these cytochromes in a subject may be responsible for the metabolization of cyclobenzaprine or amitriptyline at different rates. For subjects having an allele identified to metabolize cyclobenzaprine rapidly, a higher dose of cyclobenzaprine, in the range of about 5.0-30 mg/day, is administered. Such as from about 5.0-20 mg/day, or from about 10.0-30.0 mg/day, or from about 20.0-30.0 mg/day. For subjects having an allele identified to metabolize cyclobenzaprine more slowly, a lower dose of about 5.6 mg/day or less of cyclobenzaprine is administered, such as between about 0.1-5.0 mg/day, or from about 1.0-3.0 mg/day, or from about 3.0-5.6 mg/day. For subjects having an allele identified to metabolize amitriptyline rapidly, a higher dose of amitriptyline, in the range of about 11.0-90 mg/day, is administered. Such as from about 11.0-60 mg/day, or from about 20.0-60.0 mg/day, or from about 40.0-60.0 mg/day. For subjects having an allele identified to metabolize amitriptyline more slowly, a lower dose of about 11.2 mg/day or less of amitriptyline is administered, such as between about 1.0-11.2 mg/day, or from about 1.0-9.0 mg/day, or from about 3.0-11.2 mg/day.

A history of smoking, or use of various medications, can further influence a subject's metabolism of cyclobenzaprine or amitriptyline. For instance, smoking is a strong inducer of CYP1A2, and medications such as carbamazepine, phenytoin, phenobarbital, and nevirapine are strong inducers of CYP3A4. If a subject has a history of smoking or of use of any one of these medications, their ability to metabolize cyclobenzaprine can be altered.

A subject's metabolism of cyclobenzaprine or amitriptyline may additionally be influenced if the subject has a history of using medications that block CYP1A2, CYP3A4 or CYP2D6. Medications that block CYP1A2 include but are not limited to artemisinin, atazanavir, climetidine, ciprofloxacin, enoxacin, ethinyl estradiol, fluvoxamine, mexiletine, tacrine thiabendazole and zileuton. If a subject has a history of using any one of these medications, their ability to metabolize cyclobenzaprine or amitriptyline may be altered.

A subject having a history of smoking is a subject who currently smokes, or has been smoking for at least 1 year, or at least 2 years, or at least 3 years, or at least 4 years, or at least 5 years or at least 10 years.

In some aspects of this disclosure, the pharmacogenetic test and the subject's history of smoking and us of medications such as carbamazepine, phenytoin, phenobarbital, and nevirapine can be used separately, or in combination, to determine the dosage of cyclobenzaprine, amitriptyline or pharmaceutically acceptable salts thereof to administer to the subject. For subjects having alleles corresponding to a high cyclobenzaprine metabolizer genotype of any one of CYP3A4, CYP1A2 or CYP2D6 or a history of smoking or use of other medications including carbamazepine, phenytoin, phenobarbital, and nevirapine, the dose of cyclobenzaprine administered to the subject is greater than 5 mg/day. For subjects who do not have a high metabolizer genotype or a history of smoking or use of other medications, the dose of cyclobenzaprine administered to the subject is 5.6 mg/day or less.

The following examples are set forth as being representative of the present application. These examples are not to be construed as limiting the scope of the disclosures these and other equivalent embodiments will be apparent in view of the present disclosure, figures, and accompanying embodiments and aspects.

### EXAMPLES

### Example 1. Cyclobenzaprine Sublingual Formulation TNX-102 SL

TNX-102 SL is a sublingual formulation which contains a eutectic of cyclobenzaprine hydrochloride (the active ingredient) and D-mannitol. The formulation also contains potassium salt, dibasic. Table 1 shows the specific composition of the TNX-102 SL tablet.

TABLE 1
TNX-102 SL Sublingual Tablet Composition

| | | | Composition | |
|---|---|---|---|---|
| | Quality | | mg per | |
| Ingredient | Standard | Function | Tablet | Percent |
| Cyclobenzaprine hydrochloride | USP | Active ingredient | 2.80^{c} | 7.37% |
| Mannitol a | USP, Ph. Eur., JP | Diluent | 2.50 | 6.58% |
| Dye D&C Yellow 10 Lake | FDA approved per | Colorant | 0.023 | 0.06% |
| | 21CFR (Section | | | |
| | 74.1710) | | | |
| Mannitol/corn starch | DMF No. 23720. | Diluent | 27.977 | 73.62% |
| (Pearlitol ® Flash) ^{b} | | | | |
| Crospovidone | USP, Ph. Eur., JP | Disintegrant | 2.00 | 5.26% |
| Colloidal silica | USP, Ph. Eur., JP | Glidant | 0.50 | 1.32% |
| Sodium stearyl fumarate | NF, Ph. Eur., JP | Lubricant | 1.00 | 2.63% |
| Potassium phosphate, dibasic | USP, Ph. Eur. | pH control | 1.20 | 3.16% |

| | | |
|---|---|---|
| Total | 38.00 | 100.00% |

a Mannitol: about 0.7 mg of the 2.5 mg total amount is a component of the eutectic and the rest is diluent.
^{b} Pearlitol ® Flash is the trade name for an excipient containing about 80% mannitol and 20% corn starch.
^{c}Calculated as the HCl salt

### Example 2. Efficacy of TNX-102 SL for the Treatment of PTSD

Two 12-week, multicenter, randomized, double-blind, placebo-controlled, fixed-dose trials (P201 and P301) were conducted to investigate the efficacy and safety of sublingual cyclobenzaprine formulation (TNX-102 SL). Both trials required PTSD DSM-5 Criterion A trauma(s) incurred during military service since 2001; free of antidepressants ≥2 months; free of or washed off of other psychotropics. Both excluded severe suicide risk (intent or plan; attempt within 1 year); substance use disorders (SUDs) within 6 months; lifetime bipolar, psychotic, obsessive-compulsive, or antisocial personality disorders.

The trials analyzed the change from baseline in the severity of PTSD symptoms as measured by the Clinician-Administered PTSD Scale for DSM-5 (CAPS-5) between subjects treated with a sublingual cyclobenzaprine formulation (TNX-102 SL, 5.6 mg) and those receiving placebo over the course of 12 weeks of treatment. Subjects participating in the study were interviewed after 2 weeks, 4 weeks, 8 weeks and 12 weeks for assessment of treatment efficacy and safety.

Analysis of Subgroups with Index Trauma <9 Years and >9 Years Before Study

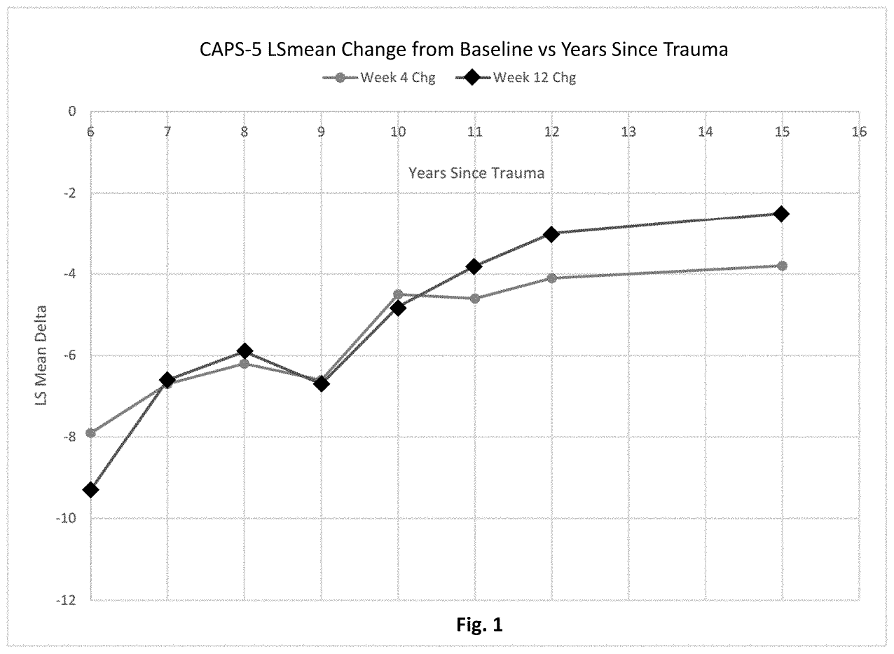

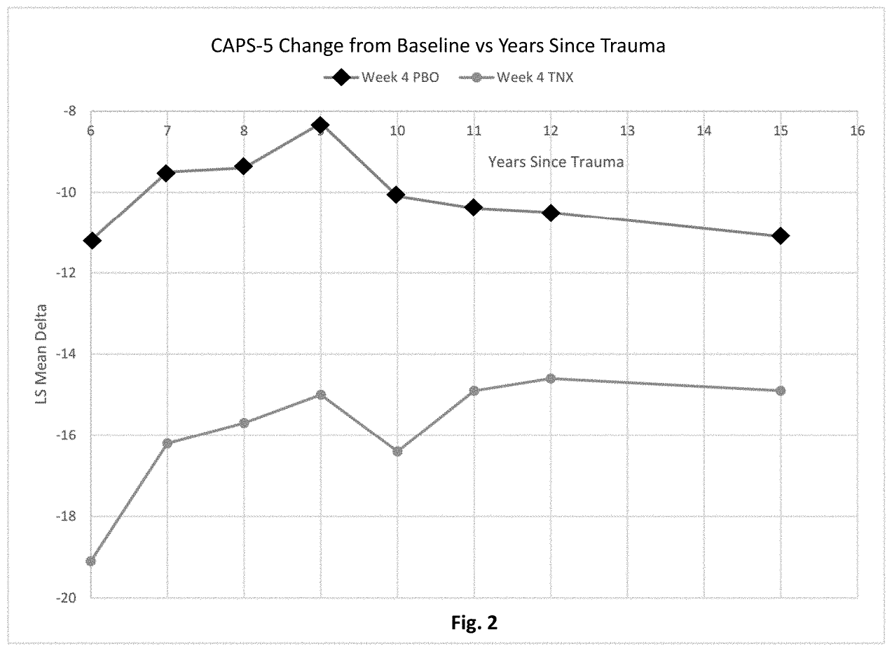

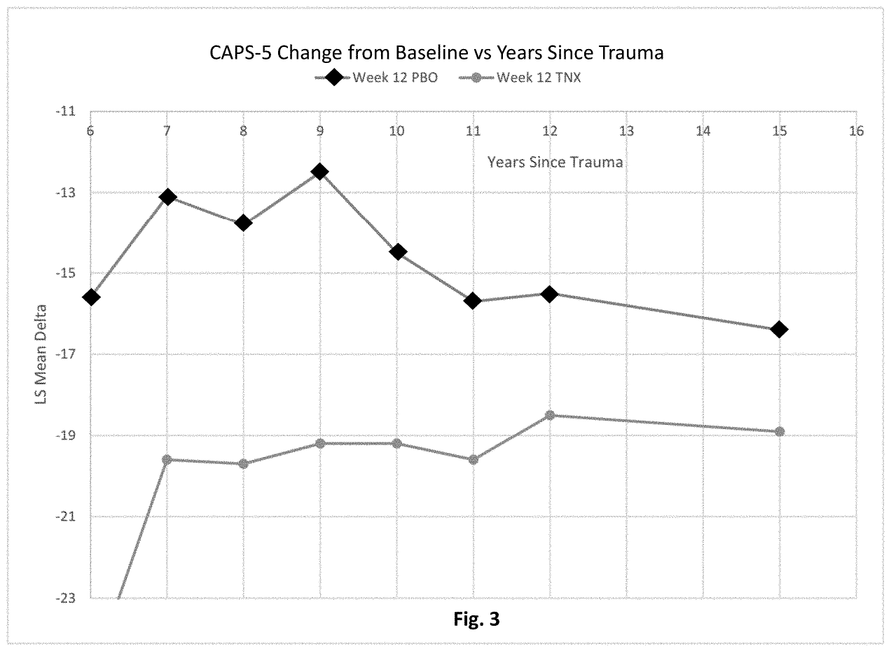

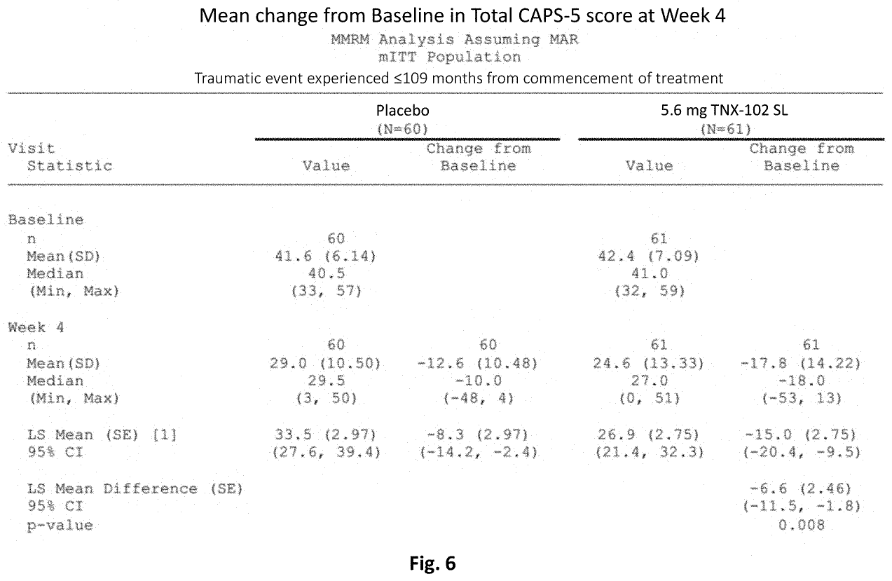

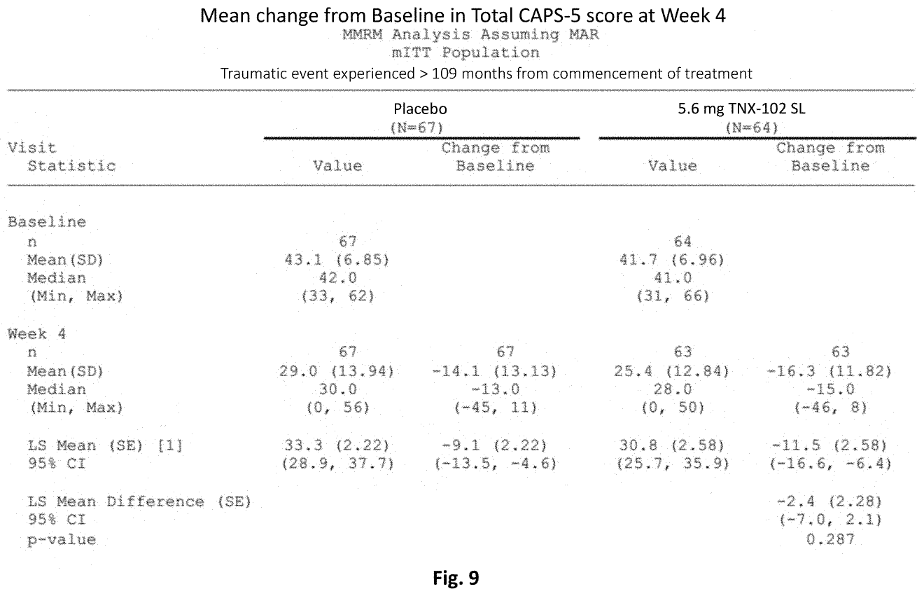

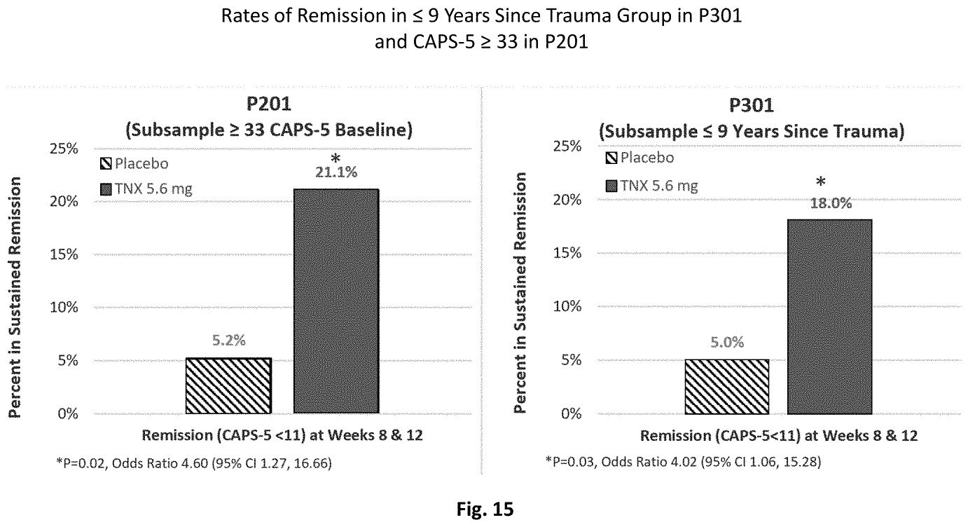

The efficacy of treatment with TNX-102 SL was found to be related to the amount of time that had passed since the incident trauma (FIGS. 1-3). Specifically, the efficacy of the treatment was highest in patients that had experienced trauma less than about 9 years prior to the start of treatment with TNX-102 SL, with the effect increasing rapidly with decreasing time since trauma. Patients that had experienced trauma more than about 9 years before the start of the trial did not show a significant benefit from the treatment. For example, as demonstrated in FIG. 6, patients that experienced a PTSD-causing trauma less than or equal to 109 months (˜9 years) prior to the start of treatment had a decrease, on average, of 6.6 points in their CAPS-5 score after 4 weeks of treatment as compared to placebo (p-value=0.008). Conversely, as demonstrated in FIG. 9, subjects receiving 4 weeks of treatment who had experienced trauma greater than 109 months (˜9 years) prior to the start of treatment did not show a significant improvement in CAPS-5 scores as compared to placebo (p-value=0.287). The rate of remission for those who experienced trauma less than about 9 years prior to the start of treatment with TNX-102 SL in the P301 trial was similar to the rates of remission observed in the P201 trial, wherein the median time since trauma was about 6 years (FIG. 15).

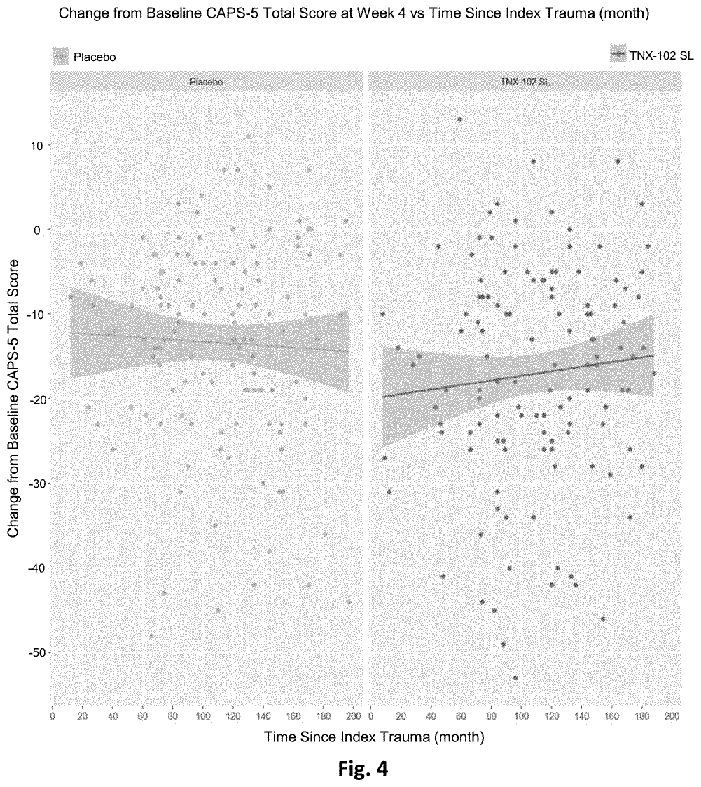

The relationship between the efficacy of the treatment according to this disclosure and the amount of time since index trauma indicates that administering cyclobenzaprine, amitriptyline or pharmaceutically acceptable salts thereof to a subject quickly after the traumatic event will be useful for subjects suffering from ASD and the prevention of PTSD. As demonstrated in FIG. 4, subjects receiving treatment sooner after experiencing a traumatic event have a greater decrease in their CAPS-5 score after 4 weeks of treatment as compared to subjects who have a longer time gap between the traumatic event and the commencement of treatment.

### Safety

There were no serious and unexpected adverse events (AEs) in trials P301 or P201. See Table 1. Observed systemic AEs were consistent with those described in approved oral cyclobenzaprine product labels. Similar severity and incidence of oral hypoaesthesia (tongue/mouth numbness) is reported across studies (37% in P301; 36% in P201) for TNX 5.6 mg.

TABLE 1
Summary of adverse events

| Categoty of | | | | | |
|---|---|---|---|---|---|
| Adverse | P201 | | | P301 | |
| Reaction | | TNX | TNX | | TNX |
| Preferred | Placebo | 2.8 mg | 5.6 mg | Placebo | 5.6 mg |
| Term | (N = 94) | (N = 93) | (N = 50) | (N = 134) | (N = 134) |

Systemic Adverse Events

| | | | | | |
|---|---|---|---|---|---|
| Somnolence | 6.4% | 11.8% | 16.0% | 9.0% | 15.7% |
| Dry Mouth | 10.6% | 4.3% | 16.0% | | |
| Headache | 4.3% | 5.4% | 12.0% | | |
| Insomnia | 8.5% | 7.5% | 6.0% | | |
| Sedation | 1.1% | 2.2% | 12.0% | | |

Local Administration Site Reaction

| | | | | | |
|---|---|---|---|---|---|
| Hypoaesthesia | 2.1% | 38.7% | 36% | 1.5% | 37.3% |
| Oral | | | | | |
| Paraesthesia | 3.2% | 16.1% | 4.0% | 0.7% | 9.7% |
| Oral | | | | | |
| Glossodynia | 1.1% | 3.2% | 6.0% | | |
| Product Taste | | | | 3.0% | 11.9% |
| Abnormal | | | | | |

Retrospective Analyses of Participants with Administration Site Reactions

TNX-102 SL is a sublingual tablet that rapidly disintegrates in the mouth and results in transmucosal absorption of cyclobenzaprine. Some local administration site reactions that occurred in TNX-102 SL treated groups more than placebo include oral numbness (ON, oral hypoaesthesia), oral tingling (OT, oral paraesthesia) and noticeable taste (NT). ON events are typically mild and transient (typically <60 min) and rarely lead to discontinuation. ON/OT/NT experiences were not elicited systematically and may have had variable reporting. ON/OT/NT events are episodic and observed infrequently. ON adverse event rate also has been consistent across studies.

To investigate the possibility of ON/OT/NT events for potential unblinding, individuals were grouped as either having experienced ON/OT/NT event or not (+ or −). In P201 and P301, experiencing ON/OT/NT event(s) seems to correlate with treatment effect based on some post hoc analyses, but not others. In P201, the TNX-102 SL 5.6 mg ON/OT/NT+ subgroup had an improvement of −6.9 points (p=0.037) relative to the improvement seen in the TNX-102 SL 5.6 mg mITT population of −4.5 points (p=0.053).

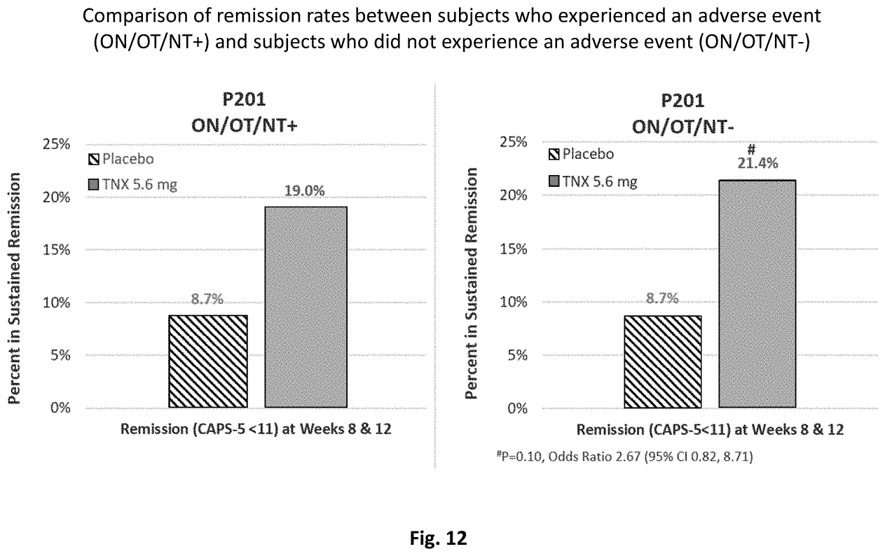

The ON/OT/NT− subgroup had a numerically lower decrease (−1.8 points; p=0.523). However, as seen in FIG. 12, in P201 sustained remission rates (CAPS-5 total <11 at both Week 8 & 12), were similar between the ON/OT/NT+ and − subgroups. In P301, the ON/OT/NT+ subgroup had an improvement in CAPS-5 of −5.5 points (p=0.010), relative to the mITT population change of −1.0 point (p=0.602). The P301 ON/OT/NT− subgroup did not improve, with a change in CAPS-5 of +1.5 points (p=0.505). In P301 the ON/OT/NT+ group appears to correlate with treatment response in the ≤9 year subsample (−13.4 points), but not in the >9 year subsample (˜0.6 points). The lack of response in the >9 year subsample that was ON/OT/NT+ indicates treatment response could not simply be due to an unblinding effect (caused by the sublingual formulation) as this subgroup would be expected to show a treatment response. Together, these findings support the interpretation that ON/OT/NT events did not account for the observed responses to TNX 5.6 mg in the P201 mITT population or the P301≤9 year subgroup.

### Treatment Effects of TNX-102 SL on Derealization in Military Related PTSD

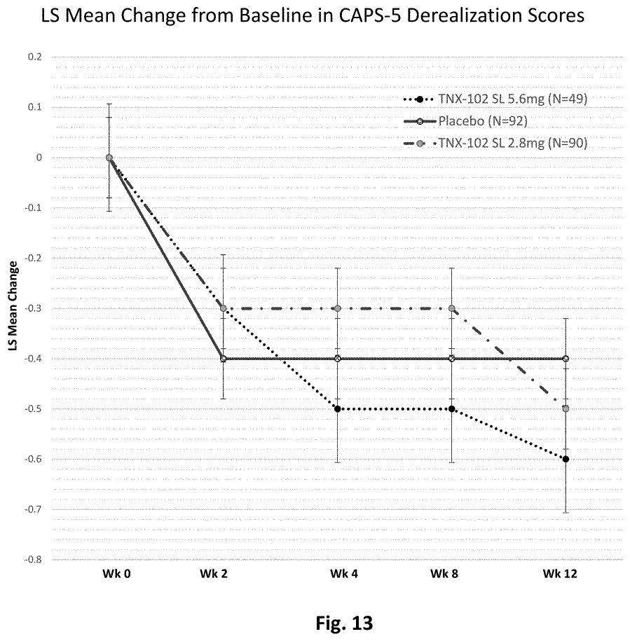

Analyses of the P201 study established that TNX-102 SL 5.6 mg is an effective treatment for derealization symptoms in the dissociative subtype by improving CAPS-5 total scores in military-related PTSD (FIG. 13). These results suggest that TNX-102 SL improved sleep in derealizers and thus decreased symptoms associated with poor sleep quality (hyperarousal and distressing dreams). This decreased their overall CAPS-5 score and allowed a significant result to be produced.

### Example 3

Cyclobenzaprine Metabolism of Subjects with a History of Smoking

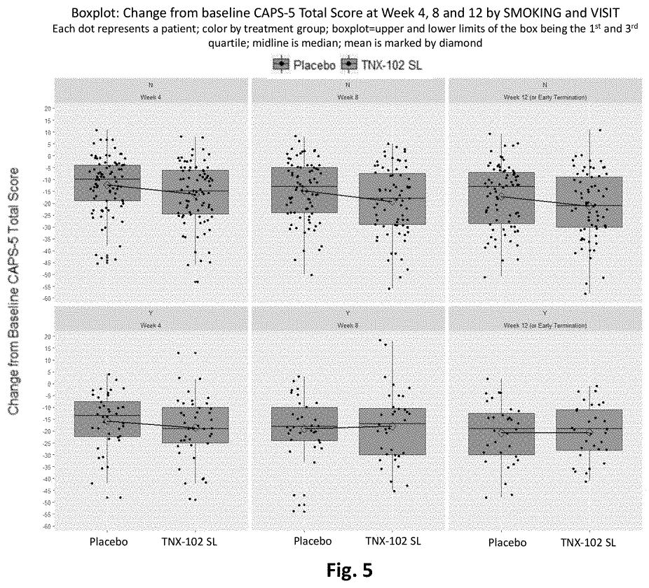

Determining a therapeutic dosage of cyclobenzaprine, amitriptyline or pharmaceutically acceptable salts thereof is important for the overall efficacy of the therapy. The therapeutic dosage may be influenced by a variety of factors, including the subject's history of smoking and use of other medications. In one aspect of this disclosure, subjects with a history of smoking had a diminished response to treatment with TNX-102 SL. As depicted in FIG. 5, after 4 weeks of treatment, subjects with a history of smoking (bottom) had a decrease in their CAPS-5 scores relative to those receiving placebo. However, this was to a lesser extent than those who did not have a history of smoking (top). Furthermore, the subject's response to the treatment ultimately flattened out relative to placebo after 8 and 12 weeks of treatment respectively. Smoking is known to be a strong inducer of CYP1A2. Without wishing to be bound by theory, this may contribute to an increased metabolism of cyclobenzaprine, as well as amitriptyline or pharmaceutically acceptable salts thereof, which plays a critical role in maintaining effective steady-state levels of cyclobenzaprine or amitriptyline in the subject.

### Effect of CYP3A4 Inducers on Cyclobenzaprine and Amitriptyline Metabolism

In view of the detrimental effect smoking appears to have on the metabolism of cyclobenzaprine or amitriptyline or pharmaceutically acceptable salts thereof, the effect of other medications is assessed to determine if they have a similar effect on cyclobenzaprine or amitriptyline metabolism. Medications such as carbamazepine, phenytoin, phenobarbital, nevirapine are known to be strong inducers of CYP3A4. Without wishing to be bound by theory, this may detrimentally influence the metabolism of cyclobenzaprine or amitriptyline in a similar manner to that of smoking. Subjects suffering from PTSD or ASD who have a history of using carbamazepine, phenytoin, phenobarbital, or nevirapine are administered TNX-102 SL 5.6 mg once daily over a period of 12 weeks. The efficacy of the treatment is assessed every 2 weeks once treatment commences. If the treatment response fails to manifest or fails to produce remission of at least one symptom by the end of the 12^{th} week, the dose of cyclobenzaprine or amitriptyline is increased. The efficacy of the higher dose is similarly assessed every 2 weeks.

Similarly to the effect CYP3A4 inducers have on increasing cyclobenzaprine or amitriptyline metabolism, the use of medications that block CYP1A2, CYP2D6 and CYP3A4 my result in a decreased rate of cyclobenzaprine or amitriptyline metabolism. Medications that block CYP1A2 include artemisinin, atazanavir, climetidine, ciprofloxacin, enoxacin, ethinyl estradiol, fluvoxamine, mexiletine, tacrine thiabendazole and zileuton. For subjects having a history of taking any one of these medications, the dose of cyclobenzaprine or pharmaceutically acceptable salts thereof administered to the subject is less than or equal to about 5.6 mg daily. Similarly, if the patient has a history of taking medications that block CYP1A2, CYP2D6 and CYP3A4, the dose of amitriptyline or pharmaceutically acceptable salts thereof administered to the subject is less than or equal to about 11.2 mg daily.

### REFERENCES

1. Kessler, R. C. et al. (1995) Posttraumatic Stress Disorder in the National Comorbidity Survey. Arch Gen Psychiatry. 52, pp. 1048-1060.
2. Goldstein, R. B. et al. (2016) The Epidemiology of DSM-5 Posttraumatic Stress Disorder in the United States: Results from the National Epidemiologic Survey on Alcohol and Related Conditions-III. Soc Psychiatry Psychiatr Epidemiol. 51 (8), pp. 1137-1148.
3. Shalev, A. Y. et al. (2012) Prevention of Posttraumatic Stress Disorder by Early Treatment. Arch Gen Psychiatry. 69 (2), pp 166-76.
4. Tucker, P. et al. (2001) Paroxetine in the Treatment of Chronic Posttraumatic Stress Disorder: Results of a Placebo-Controlled, Flexible-Dosage Trial. J Clin Psychiatry. 62 (11), pp 860-868.
5. Moldofsky H. et al. (2011) Effects of bedtime very low dose cyclobenzaprine on symptoms and sleep physiology in patients with fibromyalgia syndrome: a double-blind randomized placebo-controlled study. J Rheumatol. December; 38(12):2653-63.
6. Moldofsky H. et al. (2015) Relationship of Sleep Quality and Fibromyalgia Outcomes in a Phase 2b Randomized, Double-Blind, Placebo-Controlled Study of Bedtime, Rapidly Absorbed, Sublingual Cyclobenzaprine (TNX-102 SL). Arthritis Rheumatol.; 67 (suppl 10).
7. Katz W A and Dube J. (1988) Cyclobenzaprine in the treatment of acute muscle spasm: review of a decade of clinical experience. Clin Ther.; 10(2):216-28.

1. A method for treating post-traumatic stress disorder (PTSD) or one or more symptoms thereof in a subject in need thereof who has experienced a traumatic event less than or equal to about 9 years prior to the commencement of a treatment, wherein said treatment comprises administering to the subject a pharmaceutical composition comprising a therapeutically effective amount of cyclobenzaprine, amitriptyline, or a pharmaceutically acceptable salt thereof.

2. A method for treating acute stress disorder (ASD) or one or more symptoms thereof in a subject in need thereof who has experienced a traumatic event less than or equal to 1 month prior to the commencement of a treatment, wherein said treatment comprises administering to the subject a pharmaceutical composition comprising a therapeutically effective amount of cyclobenzaprine, amitriptyline, or a pharmaceutically acceptable salt thereof.

3. The method of claim 1 or 2, wherein the traumatic event is a DSM-5 Criterion A traumatic event.

4. The method of claim 1 or 2, wherein the pharmaceutical composition is administered once daily.

5. The method of claim 1 or 2, wherein the treatment does not exceed 4 weeks.

6. The method of claim 2, wherein the treatment of the ASD alleviates the development of PTSD and associated symptoms thereof in the subject.

7. The method of claim 1 or 2, wherein cyclobenzaprine or amitriptyline is a free base.

8. The method of claim 1 or 2, wherein the cyclobenzaprine or amitriptyline is a pharmaceutically acceptable salt thereof.

9. The method of claim 1 or 2, wherein the pharmaceutical composition is formulated for sublingual, buccal, oral, suppository, intravenous, intramuscular, subcutaneous, inhalational, intranasal, transdermal, parenteral, rectal, or vaginal administration.

10. The method of claim 9, wherein the pharmaceutical composition is formulated for sublingual administration.

11. The method of claim 1 or 2, wherein the pharmaceutical composition comprises a basifying agent.

12. The method of claim 11, wherein the basifying agent is selected from the group consisting of potassium dihydrogen phosphate, dipotassium hydrogen phosphate, tripotassium phosphate, sodium carbonate, sodium bicarbonate, calcium carbonate, calcium bicarbonate, TRIS buffer, sodium dihydrogen phosphate, disodium hydrogen phosphate, trisodium phosphate, potassium carbonate, potassium bicarbonate, potassium acetate, sodium acetate, dipotassium citrate, tripotassium citrate, disodium citrate and trisodium citrate.

13. The method of claim 1 or 2, wherein the efficacy of the treatment increases with decreasing time between experience of the traumatic event and the commencement of treatment.

14. The method of claim 1 or 2, wherein the amount of the cyclobenzaprine, or a pharmaceutically acceptable salt thereof that is administered to the subject is between about 0.1 mg/day and about 50 mg/day.

15. The method of claim 14, wherein the amount of the cyclobenzaprine or a pharmaceutically acceptable salt thereof that is administered to the subject is between about 0.5 mg/day and about 30 mg/day.

16. The method of claim 15, where the amount of the cyclobenzaprine or pharmaceutically acceptable salt thereof that is administered to the subject is between about 1 mg/day and about 20 mg/day.

17. The method of claim 1 or 2, wherein the amount of the amitriptyline, or a pharmaceutically acceptable salt thereof that is administered to the subject is between about 0.1 mg/day and about 150 mg/day.

18. The method of claim 17, wherein the amount of amitriptyline or pharmaceutically acceptable salt thereof that is administered is between about 1.0 mg/day and about 90 mg/day.

19. The method of claim 18, where the amount of the amitriptyline or a pharmaceutically acceptable salt thereof that is administered to the subject is between about 3 mg/day and about 60 mg/day.

20. The method of claim 1 or 2, wherein the pharmaceutical composition is administered to the subject either sequentially or concurrently with a compound selected from the group consisting of an alpha-1-adrenergic receptor antagonist, a beta-adrenergic antagonist, an anticonvulsant, a selective serotonin reuptake inhibitor and a serotonin-norepinephrine reuptake inhibitor or a pharmaceutical composition containing one or more of them.

21. The method of claim 20, wherein the alpha-1-adrenergic receptor antagonist is prazosin and wherein the selective serotonin reuptake inhibitor is sertraline, paroxetine, fluoxetine, citalopram or escitalopram.

22. (canceled)

23. The method of claim 1 or 2, wherein the treatment is combined with psychotherapeutic intervention.

24. The method of claim 1, wherein at least one of the symptoms of PTSD is eliminated or ameliorated.

25. The method of claim 24, wherein the symptoms of PTSD are selected from the group consisting of intrusion symptoms, avoidance symptoms, cognition and mood symptoms, arousal and reactivity symptoms, difficulty falling sleep, irritability, difficulty concentrating, hypervigilance, and persistent exaggerated startle response.

26. The method of claim 2, wherein at least one of the symptoms of ASD is eliminated or ameliorated.

27. The method of claim 26, wherein the symptoms of ASD are selected from the group consisting of reexperiencing symptoms, avoidance symptoms, arousal symptoms, difficulty with sleep, nightmares, irritability, difficulty concentrating, hypervigilance, persistent exaggerated startle response, feelings such as not knowing where you are, and feeling as if you are outside of your body.

28. The method of claim 1, wherein the treatment is during the rapid recovery phase, the remitting phase, or the persistent phase of PTSD.

29. A method for treating or preventing PTSD, ASD or one or more associated symptoms thereof in a subject in need or at risk thereof, wherein the method comprises:
a) administering to the subject a pharmaceutical composition comprising cyclobenzaprine, amitriptyline or a pharmaceutically acceptable salt thereof;
b) assessing the efficacy of the method periodically over a course of the administration of the pharmaceutical composition;
c) suspending the administration of the pharmaceutical composition when the efficacy diminishes;
d) resuming the administration of the pharmaceutical composition 4 weeks after the administration was suspended;
wherein steps (a)-(d) may be repeated one or more times.

30. A method for treating or preventing PTSD, ASD or one or more associated symptoms thereof in a subject in need or at risk thereof, wherein the method comprises:
a) administering to the subject a pharmaceutical composition comprising cyclobenzaprine, amitriptyline or a pharmaceutically acceptable salt thereof;
b) suspending the administration of the pharmaceutical composition after about 4 weeks;
c) resuming the administration of the pharmaceutical composition about 4 weeks after suspending the administration;
wherein steps (a)-(c) may be repeated one or more times.

31. The method of claim 29 or 30, wherein the treatment or prevention is of PTSD, and the subject has experienced a traumatic event less than or equal to about 9 years prior to the commencement of treatment.

32. The method of claim 29, wherein the efficacy of is measured at least about every 2 weeks after the administration of the pharmaceutical composition begins.

33. The method of claim 32, wherein the efficacy is assessed based on the subject's Clinician Administered PTSD Scale for DSM-5 (CAPS-5) score.

34-56. (canceled)

57. A method of determining a therapeutic dosage of cyclobenzaprine or a pharmaceutically acceptable salt thereof for the treatment or prevention of PTSD, ASD or one or more associated symptoms thereof in a subject in need or at risk thereof comprising:
a) obtaining a suitable cell or tissue sample from a subject suffering from PTSD or ASD or at risk therefor;
b) identifying the CYP1A2, CYP2D6, and CYP3A4 genotype of said subject to determine if the patient has a high cyclobenzaprine metabolizer genotype;
c) assessing the subject's medical history for a history of smoking or use of medications that act as inducers of CYP3A4;
wherein if the subject has at least one of the criteria identified in step (b) or (c), the dose of cyclobenzaprine administered to the subject is greater than about 5 mg/day;
wherein if the subject does not have at least one of the criteria identified in step (b) or (c), the dose of cyclobenzaprine administered to the subject is about 5.6 mg/day or less.

58. A method of determining a therapeutic dosage of amitriptyline or a pharmaceutically acceptable salt thereof for the treatment or prevention of PTSD, ASD or one or more associated symptoms thereof comprising:
a) obtaining a suitable cell or tissue sample from a subject suffering from PTSD or ASD or at risk thereof;
b) identifying the CYP1A2, CYP2D6, and CYP3A4 genotype of said subject to determine if the patient has a high amitriptyline metabolizer genotype;
c) assessing the subject's medical history for a history of smoking or use of medications that act as inducers of CYP3A4;
wherein if the subject has at least one of the criteria identified in step (b) or (c), the dose of amitriptyline administered to the subject is greater than about 11 mg/day;
wherein if the subject does not have at least one of the criteria identified in step (b) or (c), the dose of amitriptyline administered to the subject is about 11.2 mg/day or less.

59. The method of claim 57 or 58, wherein the medications that act as inducers of CYP3A4 are selected from carbamazepine, phenytoin, phenobarbital, and nevirapine.

60. The method of claim 57 or 58, wherein the subject has experienced a traumatic event less than or equal to about 9 years prior to the commencement of treatment.

61-75. (canceled)
